(19) United States
(12) Patent Application Publication
(10) Pub. No.: US 2026/0248762 A1
(43) Pub. Date: Aug. 27, 2026

(54) RRX-001 FOR MINIMIZING POST-INFARCT ADVERSE VENTRICULAR REMODELING AND COMPLICATIONS

(71) Applicant: EpicentRx, Inc., La Jolla, CA (US)

(72) Inventors: Bryan ORONSKY, Los Altos Hills, CA (US); Scott CAROEN, San Francisco, CA (US); Tony R. REID, San Diego, CA (US)

(21) Appl. No.: 19/126,990

(22) PCT Filed: Nov. 3, 2023
(86) PCT No.: PCT/US2023/078708

§ 371 Date: May 2, 2025

Related U.S. Application Data
(60) Provisional application No. 63/422,642, filed on Nov. 4, 2022.

(51) Int. Cl. A61K 31/397; A61K 35/14; A61K 35/18; A61P 9/10

(52) U.S. Cl. CPC A61K 31/397; A61K 35/14; A61K 35/18; A61P 9/10

(57) ABSTRACT

Compositions and methods for treating or preventing a condition related to myocardial infarction and infarct expansion, extension, and/or dilation following a cardiovascular disorder incident (e.g., post-myocardial infarction) using an effective amount of RRx-001, or a pharmaceutically acceptable salt thereof, are described. In some embodiments, an agent (such as a statin, an angiotension-converting-enzyme (ACE) inhibitor, aspirin, oxygen, a nitric oxide donor, a drug-eluting stent, an anticoagulant or antiplatelet therapy, a phosphodiesterase-5 inhibitor, a calcium channel blocker, an angiotensin II receptor blocker (ARB), a beta blocker, an aldosterone antagonist, a loop diuretic, an epigenetic inhibitor, and/or a nitrite) is administered prior to, concurrently with, or subsequent administering the effective amount of RRx-001, or a pharmaceutically acceptable salt thereof. In some embodiments, the effective amount of RRx-001, or a pharmaceutically acceptable salt thereof, is administered as a composition comprising a blood product and/or the agent.

### CROSS-REFERENCE TO RELATED APPLICATIONS

This application claims priority to U.S. Provisional Patent Application Ser. 63/422,642 filed on Nov. 4, 2022, the entire contents of which are hereby incorporated by reference in its entirety.

### FIELD OF THE INVENTION

This disclosure relates to compositions and methods to prevent or minimize post-infarct remodeling.

### BACKGROUND

Cardiovascular diseases, including acute myocardial infarction (MI), colloquially known as a “heart attack”, are a leading cause of death worldwide. The World Health Organization has predicted that 23.3 million people will die annually from coronary artery disease (CAD) by 2030. Examples of cardiovascular disorders that affect a significant number of patients include pulmonary hypertension, systemic hypertension, hypercholesterolemia, angina (e.g., Prinzmetal's angina), Cardiac syndrome X, acute MI, peripheral artery disease, Raynaud's disease, pulmonary embolism, and intravascular thrombosis. As an example, acute MI is a common complication of CAD and is characterized by cardiomyocyte necrosis and acute loss of myocardial tissue. The principal complications of acute MI include acute left ventricular (LV) failure, cardiogenic shock, cardiorrhexis (rupture of the heart), conduction defects, and pericarditis. See Jaffe (1984) Cardiol. Clin. 2(1):79-94. Massive cell death leads to an inflammatory response that is dominated by an influx of neutrophils, and which is necessary for wound healing, but is also harmful due to infarct expansion.

Two complications of acute MI that that occur early on are infarct expansion and infarct extension. Infarct expansion, disproportionate thinning, and dilatation of the infarct segment begin within hours of acute infarction and usually reach peak extent within seven to 14 days. See H. Weisman, et al. (1987) Prog Cardiovasc Dis 30(2) 73-110. Both increase the functional infarct size, the former through thinning, stretching, and dilation of the infarct region, and the latter through reinfarction, which leads to an increase in total mass of infarcted tissue.

When an MI occurs, the myocardial tissue that is no longer receiving adequate blood flow dies and is replaced with scar tissue. This infarcted tissue cannot contract during systole and may undergo lengthening in systole that leads to an immediate depression in ventricular function. This abnormal motion of the infarcted tissue can cause delayed conduction of electrical activity to the still surviving peri-infarct tissue and also places extra mechanical stress on the peri-infarct tissue. These factors individually and in combination contribute to the eventual myocardial dysfunction observed in the myocardial tissue remote from the site of the infarction.

At present, rapid and safe restoration of blood supply to ischemic myocardium is considered the best and the most effective treatment for acute MI. However, revascularization may also aggravate myocardial damage, produce a second blow to the myocardium, and cause myocardial reperfusion injury after ischemia.

To preserve cardiac function and minimize the diastolic and systolic wall stress caused by MI, the structural and biomechanical response in the heart leads to several changes, including collagen deposition with scar formation, fibrosis, hypertrophy, and modifications in the architecture of the left ventricle. All these changes are associated with ventricular remodeling, which affects ventricle function after MI, ultimately increasing the risk of heart failure. Post-MI cardiac remodeling is associated with ischemia/reperfusion injury, inflammation, cell death, and deposition of extracellular matrix (ECM).

Although several therapies are routinely prescribed post-MI including angiotensin converting enzyme (ACE) inhibitors, angiotensin receptor blockers (ARB), aldosterone antagonists, and β-adrenergic receptor blockers (β-blockers), they do not necessarily slow heart failure progression or reduce mortality. See Ferrario (2016) Ther. Adv. Cardiovasc. Dis. 10(3):162-171. Since post-MI cardiac remodeling directly impacts not only the recovery of cardiac functions but also the survival rate, there is an urgent need in the art for treatments that target post-MI cardiac remodeling.

The present disclosure relates to methods for cardioprotection following a cardiovascular disorder incident in subjects. Past research efforts have led to significant advances in the detection, evaluation, and treatment of cardiovascular disorders. However, despite these developments, these medical conditions remain significant health problems for many patients. Accordingly, there is need for new therapeutic methods for cardioprotection following a cardiovascular disorder incident. The present invention addresses this need and provides other related advantages.

### SUMMARY

An embodiment of the disclosed subject matter includes a method for treating or preventing a condition related to myocardial infarction and infarct expansion, extension, and/or dilatation and adverse remodeling following a cardiovascular disorder incident in a subject. The method includes administering an effective amount of RRx-001, or a pharmaceutically acceptable salt thereof, to the subject at a time period post-infarction.

In some embodiments, the method is used for preventing cardiac remodeling after myocardial infarction. In some embodiments, the method is used for preventing cardiac dilatation after myocardial infarction. In some embodiments, the method is used for improving cardiac function after myocardial infarction. In some embodiments, the method is used for preventing heart failure after myocardial infarction.

In some embodiments, administering a therapeutically effective amount of the RRx-001, or a pharmaceutically acceptable salt thereof, improves cardiac function in the subject experiencing at least one of systolic and/or diastolic dysfunction of the left ventricle (LV) and/or right ventricle (RV), myocardial hypertrophy, myocardial dilatation, myocardial fibrosis, depressed myocardial shortening, diminished cardiac contractility, reduced stroke volume, diminished fractional shortening, diminished ejection fraction, increased LV end diastolic pressure, increased ventricular wall stress, increased ventricular wall tension, increased LV systolic volume, increased LV diastolic volume, and/or increased thickness of the posterior wall of the heart.

In some embodiments, the cardiac remodeling results from an acute coronary syndrome (e.g., ST elevation myocardial infarction (STEMI) or non-ST elevation acute coronary syndrome (NSTE-ACS)), cardiac fibrosis, a pulmonary embolism, pericarditis, myocarditis, coronary vasospasm, hypertension, myocardial infarction, a valvular heart disease (e.g., from a mechanical prosthetic valve, mitral rheumatic stenosis, infectious endocarditis, or marantic endocarditis), left ventricular dysfunction, cardiac ischemia, angina, tachycardia, bradycardia, hypertrophic cardiomyopathy, aortic dissection, aortitis, dilated myocardiopathy, a heart valve disease, left ventricular hypertrophy, atrial myxoma, and/or a congenital heart diseases (such as patent foramen ovale, atrial septal aneurysm and/or ventricular septal defects).

The effective amount of the RRx-001, or a pharmaceutically acceptable salt thereof, may be a therapeutically effective amount of the RRx-001, or a pharmaceutically acceptable salt thereof. In certain embodiments, the RRx-001, or a pharmaceutically acceptable salt thereof, is administered at a daily dosage of from about 0.1 g/kg to about 10 mg/kg, about 1 g to about 5 mg/kg, about 0.05 mg/kg to about 10 mg/kg, about 0.1 mg/kg to about 10 mg/kg, about 0.1 mg/kg to about 5 mg/kg, about 0.5 mg/kg to about 5 mg/kg, about 0.5 mg/kg to about 3 mg/kg, about 0.1 mg/kg to about 1.5 mg/kg, about 0.1 mg/kg to about 0.35 mg/kg, about 0.35 mg/kg to about 0.75 mg/kg, or about 0.75 mg/kg to about 1 mg/kg. In preferred embodiments, the therapeutically effective amount of RRx-001, or a pharmaceutically acceptable salt thereof, is about 0.5 mg/kg.

In some embodiments, the time period post-infarction is in a range of about 1 hour to about three months. In some embodiments, the time period post-infarction is in the range of about one hour to about one month. In some embodiments, the time period post-infarction is in the range of about one hour to about 21 days. In some embodiments, administering the effective amount of RRx-001, or a pharmaceutically acceptable salt thereof, is performed via a single administration. In some embodiments, the subject is a mammal subject, and preferably a human subject. In some embodiments, the subject is an animal subject.

In some embodiments, administering the therapeutically effective amount of RRx-001, or a pharmaceutically acceptable salt thereof, is performed via at least two administrations. In some embodiments, administering the therapeutically effective amount of RRx-001, or a pharmaceutically acceptable salt thereof, is performed at a frequency of between about 1 time per hour and about 1 time per month during the time period. In some embodiments, administering the therapeutically effective amount of RRx-001, or a pharmaceutically acceptable salt thereof, is performed at a frequency of between about 4 times per day and about 1 time per week during the time period. In some embodiments, administering the therapeutically effective amount of RRx-001, or a pharmaceutically acceptable salt thereof, is performed at a frequency of between about 2 times per day and about 3 times per week during the time period. In some embodiments, administering the therapeutically effective amount of RRx-001, or a pharmaceutically acceptable salt thereof, is performed at a frequency of about one time a day during the time period. In some embodiments, administering the therapeutically effective amount of RRx-001, or a pharmaceutically acceptable salt thereof, is performed at a frequency of between about 1 time per day and about 2 times per week during the time period.

In some embodiments, administering the effective amount of RRx-001, or a pharmaceutically acceptable salt thereof, occurs via oral administration, transdermal administration, administration by inhalation, nasal administration, topical administration, intra-aural administration, rectal administration, intravenous administration, intramuscular administration, subcutaneous administration, elution from a stent, or intraperitoneal administration, or combinations thereof. In some embodiments, administering the effective amount of the RRx-001, or a pharmaceutically acceptable salt thereof, occurs parenterally. In other embodiments, administering the effective amount of the RRx-001, or a pharmaceutically acceptable salt thereof, occurs orally. In some embodiments, administering the effective amount of the RRx-001, or a pharmaceutically acceptable salt thereof, occurs percutaneously. In some embodiments, administering the effective amount of the RRx-001, or a pharmaceutically acceptable salt thereof, occurs epicardially. In some embodiments, administering the effective amount of the RRx-001, or a pharmaceutically acceptable salt thereof, occurs endocardially. In some embodiments, administering the effective amount of the RRx-001, or a pharmaceutically acceptable salt thereof, occurs via intravenous administration. In some embodiments, administering the effective amount of the RRx-001, or a pharmaceutically acceptable salt thereof, occurs via direct intratumoral injection.

In some embodiments, the cardiovascular disorder is selected from the group consisting of: pulmonary hypertension, systemic hypertension, angina (e.g., Prinzmetal's angina), Cardiac syndrome X, acute MI, stroke, aneurysm, atrial fibrillation, congestive heart failure, dilated myocardiopathy, a heart valve disease (such as mechanical prosthetic valve, mitral rheumatic stenosis, infectious endocarditis, and marantic endocarditis), left ventricular hypertrophy, atrial myxoma and congenital heart diseases (such as tetralogy of Fallot, patent foramen ovale (PFO) or atrial septal defect (ASD), atrial septal aneurysm, and ventricular septal defects peripheral artery disease), hypercholesterolemia, diabetes mellitus, Raynaud's disease, pulmonary embolism, or intravascular thrombosis. In preferred embodiments, the cardiovascular disorder is acute myocardial infarction.

In some embodiments, the condition related to myocardial infarction and infarct expansion and/or extension is selected from the group consisting of: atherosclerosis, coronary artery spasm, a chronic disease (e.g., human immunodeficiency virus (HIV), diabetes, hypothyroidism, hyperthyroidism, hemochromatosis, or amyloidosis), thrombosis, an embolism, and stenosis.

In some embodiments, the method is for preventing cardiac dilatation after myocardial infarction. In some embodiments, the method is for improving cardiac function after myocardial infarction. In some embodiments, the method is for preventing heart failure after myocardial infarction. In some embodiments, the method is for preventing cardiac remodeling after myocardial infarction.

In some embodiments, administering the therapeutically effective amount of RRx-001, or a pharmaceutically acceptable salt thereof, preserves heart ejection fraction and fractional shortening and mitigates an increase in left ventricular end-systolic diameter (LVESD) and left ventricular end-diastolic diameter (LVEDD). In some embodiments, administering the therapeutically effective amount of RRx-001, or a pharmaceutically acceptable salt thereof, preserves myocardial tissue. In some embodiments, administering the therapeutically effective amount of RRx-001, or a pharmaceutically acceptable salt thereof, reduces a level of at least one serum biomarker in the subject post-infarction. In some embodiments, each serum biomarker of the at least one serum biomarker is selected from the group consisting of: lactate dehydrogenase (LDH), creatine phosphokinase-MB (CK-MB), and cardiac Troponin 1 (cTn1). In some embodiments, administering the therapeutically effective amount of RRx-001, or a pharmaceutically acceptable salt thereof, reduces at least one of: infarct expansion, infarct scarring, myocardial hypertrophy, atrial fibrosis, collagen, IL-1β level, IL-18 level, caspase 3 enzyme activity, cardiomyocytes necrosis, inflammatory cell infiltration, and fibrous hyperplasia in the subject.

In some embodiments, the therapeutically effective amount of the RRx-001, or a pharmaceutically acceptable salt thereof, is administered as a composition comprising a blood product. In some embodiments, the blood product comprises erythrocyte cells. In some embodiments, the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis. In some embodiments, the blood product is a mixture of packed red blood cells. In some embodiments, the blood product is whole blood. In some embodiments, the whole blood is autologous whole blood or donor-matched allogenic whole blood.

In some embodiments, the method further comprises administering an agent prior to, concurrently with, or subsequent administering the effective amount of RRx-001, or a pharmaceutically acceptable salt thereof. In some embodiments, the agent is selected from the group consisting of: a statin, an angiotensin-converting-enzyme (ACE) inhibitor, aspirin, oxygen, a nitric oxide donor, a drug-eluting stent, an anticoagulant or antiplatelet therapy, a phosphodiesterase-5 inhibitor, a calcium channel blocker, an angiotensin II receptor blocker (ARB), a beta blocker, an aldosterone antagonist, a loop diuretic, an epigenetic inhibitor, and a nitrite.

In some embodiments, the effective amount of RRx-001, or a pharmaceutically acceptable salt thereof, is combined with a statin. In some embodiments, the effective amount of RRx-001, or a pharmaceutically acceptable salt thereof, is combined with an ACE inhibitor. In some embodiments, the effective amount of RRx-001, or a pharmaceutically acceptable salt thereof, is combined with an ARB. In some embodiments, the effective amount of RRx-001, or a pharmaceutically acceptable salt thereof, is combined with a beta blocker. In some embodiments, the effective amount of RRx-001, or a pharmaceutically acceptable salt thereof, is combined with an aldosterone antagonist. In some embodiments, the effective amount of RRx-001, or a pharmaceutically acceptable salt thereof, is combined with a loop diuretic. In some embodiments, the effective amount of RRx-001, or a pharmaceutically acceptable salt thereof, is combined with an epigenetic inhibitor. In some embodiments, the effective amount of RRx-001, or a pharmaceutically acceptable salt thereof, is combined with nitrite. In some embodiments, an amount of the agent is in a range of about 1 mg to about 50 mg. In some embodiments, the amount of the agent is in the range of about 1 mg to about 15 mg.

In some embodiments, administering the agent prior to, concurrently with, or subsequent administering the effective amount of RRx-001, or a pharmaceutically acceptable salt thereof, prevents at least one side effect associated with the agent. In some embodiments, administering the agent prior to, concurrently with, or subsequent administering the effective amount of RRx-001, or a pharmaceutically acceptable salt thereof, reduces at least one side effect associated with the agent.

Another embodiment of the present invention describes a pharmaceutical composition for treating or preventing a condition related to myocardial infarction and infarct expansion, extension, and/or dilatation and adverse remodeling following a cardiovascular disorder incident in a subject. The pharmaceutical composition comprises an effective amount of RRx-001, or a pharmaceutically acceptable salt thereof and may optionally comprise a blood product and/or at least one agent.

Various enumerated embodiments are contemplated herein. For example, in Embodiment 1, a method for treating, preventing, or preventing progression of a condition related to myocardial infarction and infarct expansion and/or extension following a cardiovascular disorder incident in a subject is described. The method includes administering an effective amount of RRx-001, alone or in combination, to the subject at a time period post-infarction.

Embodiment 2. The method of Embodiment 1 for preventing cardiac remodeling after myocardial infarction.

Embodiment 3. The method of Embodiment 1 for preventing cardiac dilatation after myocardial infarction.

Embodiment 4. The method of Embodiment 1 for improving cardiac function after myocardial infarction.

Embodiment 5. The method of Embodiment 1 for preventing heart failure after myocardial infarction.

Embodiment 6. The method of Embodiment 1, wherein cardiac function is improved in the subject experiencing one or more of systolic and/or diastolic dysfunction of the left ventricle (LV) and/or right ventricle (RV), myocardial hypertrophy, myocardial dilatation, myocardial fibrosis, depressed myocardial shortening, diminished cardiac contractility, reduced stroke volume, diminished fractional shortening, diminished ejection fraction, increased LV end diastolic pressure, increased ventricular wall stress, increased ventricular wall tension, increased LV systolic volume, increased LV diastolic volume, and increased thickness of the posterior wall of the heart.

Embodiment 7. The method of Embodiment 1, wherein cardiac remodeling results from one or more conditions consisting of: acute coronary syndromes e.g., ST elevation myocardial infarction (STEMI); non-ST elevation acute coronary syndrome (NSTE-ACS), cardiac fibrosis, pulmonary embolism, pericarditis, myocarditis, coronary vasospasm, hypertension, myocardial infarction, valvular heart disease from a mechanical prosthetic valve, mitral rheumatic stenosis, infectious endocarditis, or marantic endocarditis, left ventricular dysfunction, cardiac ischemia, angina, tachycardia, bradycardia, hypertrophic cardiomyopathy, aortic dissection and aortitis. dilated myocardiopathy, heart valve disease, left ventricular hypertrophy, atrial myxoma and congenital heart diseases such as patent foramen ovale, atrial septal aneurysm and ventricular septal defects.

Embodiment 8. The method of Embodiment 1, wherein the effective amount of RRx-001, or the pharmaceutically acceptable salt thereof, is a therapeutically effective amount of the RRx-001, or the pharmaceutically acceptable salt thereof.

Embodiment 9. The method of Embodiment 8, wherein the therapeutically effective amount of the RRx-001, or the pharmaceutically acceptable salt thereof, is in a range of about 0.1 g/kg to about 10 mg/kg.

Embodiment 10. The method of Embodiment 9, wherein the therapeutically effective amount of the RRx-001, or the pharmaceutically acceptable salt thereof, is in a range of about 1 g to about 5 mg/kg.

Embodiment 11. The method of Embodiment 9, wherein the therapeutically effective amount of the RRx-001, or the pharmaceutically acceptable salt thereof, is in a range of about 0.05 mg/kg to about 10 mg/kg.

Embodiment 12. The method of Embodiment 9, wherein the therapeutically effective amount of the RRx-001, or the pharmaceutically acceptable salt thereof, is in a range of about 0.1 mg/kg to about 5 mg/kg.

Embodiment 13. The method of Embodiment 9, wherein the therapeutically effective amount of the RRx-001, or the pharmaceutically acceptable salt thereof, is in a range of about 0.5 mg/kg to about 5 mg/kg.

Embodiment 14. The method of Embodiment 9, wherein the therapeutically effective amount of the RRx-001, or the pharmaceutically acceptable salt thereof, is in a range of about 0.5 mg/kg to about 3 mg/kg.

Embodiment 15. The method of Embodiment 9, wherein the therapeutically effective amount of the RRx-001, or the pharmaceutically acceptable salt thereof, is in a range of about 0.1 mg/kg to about 1.5 mg/kg.

Embodiment 16. The method of Embodiment 9, wherein the therapeutically effective amount of the RRx-001, or the pharmaceutically acceptable salt thereof, is in a range of about 0.1 mg/kg to about 0.35 mg/kg.

Embodiment 17. The method of Embodiment 9, wherein the therapeutically effective amount of the RRx-001, or the pharmaceutically acceptable salt thereof, is in a range of about 0.35 mg/kg to about 0.75 mg/kg.

Embodiment 18. The method of Embodiment 9, wherein the therapeutically effective amount of the RRx-001, or the pharmaceutically acceptable salt thereof, is in a range of about 0.75 mg/kg to about 1 mg/kg.

Embodiment 19. The method of Embodiment 1, wherein the time period post-infarction is in a range of about 1 hour to 21 days.

Embodiment 20. The method of Embodiment 1, wherein administering the effective amount of RRx-001, or the pharmaceutically acceptable salt thereof, is performed via a single administration.

Embodiment 21. The method of Embodiment 1, wherein the subject is a mammal subject.

Embodiment 22. The method of Embodiment 21, wherein the mammal subject is a human subject.

Embodiment 23. The method of Embodiment 1, wherein administering the effective amount of the RRx-001, or the pharmaceutically acceptable salt thereof, occurs parenterally.

Embodiment 24. The method of Embodiment 1, wherein administering the effective amount of the RRx-001, or the pharmaceutically acceptable salt thereof, occurs orally.

Embodiment 25. The method of Embodiment 1, wherein administering the effective amount of the RRx-001, or the pharmaceutically acceptable salt thereof, occurs percutaneously.

Embodiment 26. The method of Embodiment 1, wherein administering the effective amount of the RRx-001, or the pharmaceutically acceptable salt thereof, occurs epicardially.

Embodiment 27. The method of Embodiment 1, wherein administering the effective amount of the RRx-001, or the pharmaceutically acceptable salt thereof, occurs endocardially.

Embodiment 28. The method of Embodiment 1, wherein the cardiovascular disorder is selected from the group consisting of: pulmonary hypertension, systemic hypertension, angina (e.g., Prinzmetal's angina), Cardiac syndrome X, acute MI, stroke, aneurysm, atrial fibrillation, congestive heart failure, dilated myocardiopathy, heart valve disease such as mechanical prosthetic valve, mitral rheumatic stenosis, infectious endocarditis, and marantic endocarditis, left ventricular hypertrophy, atrial myxoma and congenital heart diseases such as tetralogy of Fallot, patent foramen ovale (PFO) or atrial septal defect (ASD), atrial septal aneurysm and ventricular septal defects peripheral artery disease, Raynaud's disease, pulmonary embolism, or intravascular thrombosis.

Embodiment 29. The method of Embodiment 28, wherein the cardiovascular disorder is acute myocardial infarction.

Embodiment 30. The method of any of the Embodiments 1-29, wherein RRx-001 is combined with a statin.

Embodiment 31. The method of any of the Embodiments 1-29, wherein RRx-001 is combined with an ACE inhibitor.

Embodiment 32. The method of any of the Embodiments 1-29, wherein RRx-001 is combined with an ARB.

Embodiment 33. The method of any of the Embodiments 1-29, wherein RRx-001 is combined with a beta blocker.

Embodiment 34. The method of any of the Embodiments 1-29, wherein RRx-001 is combined with an aldosterone antagonist.

Embodiment 35. The method of any of the Embodiments 1-29, wherein RRx-001 is combined with a loop diuretic.

Embodiment 36. The method of any of the Embodiments 1-29, wherein RRx-001 is combined with an epigenetic inhibitor.

Embodiment 37. The method of any of the Embodiments 1-29, wherein RRx-001 is combined with nitrite.

### BRIEF DESCRIPTION OF THE DRAWINGS

FIG. 1 depicts a graph comparing acute changes in heart rate (HR) during reperfusion for various groups of Example 1, according to at least some embodiments disclosed herein.

FIG. 2A depicts a graph showing echocardiographic results associated with heart ejection fractions (EFs) for various groups of Example 1, according to at least some embodiments disclosed herein.

FIG. 2B depicts a graph showing echocardiographic results associated with fractional shortenings (FSs) for various groups of Example 1, according to at least some embodiments disclosed herein.

FIG. 2C depicts a graph showing echocardiographic results associated with early relaxation velocities (E′) for various groups of Example 1, according to at least some embodiments disclosed herein.

FIG. 2D depicts a graph showing echocardiographic results associated with an E′/E ratio of diastolic function for various groups of Example 1, according to at least some embodiments disclosed herein.

FIG. 3 depicts a graph showing a myocardial infarct area (MI) for various groups of Example 1, according to at least some embodiments disclosed herein.

FIG. 4 depicts images showing a degree of damaged myocardium and fibrotic scar tissue of Example 1, according to at least some embodiments disclosed herein.

FIG. 5A depicts a graph showing RRx-001 reduced levels of lactate dehydrogenase (LDH) post-IR of Example 1, according to at least some embodiments disclosed herein.

FIG. 5B depicts a graph showing RRx-001 reduced levels of creatine phosphokinase-MB (CK-MB) post-IR of Example 1, according to at least some embodiments disclosed herein.

FIG. 5C depicts a graph showing RRx-001 reduced levels of cardiac Troponin 1 (cTn1) post-IR of Example 1, according to at least some embodiments disclosed herein.

FIG. 6 depicts images showing hematoxylin and eosin (H&E) staining and Masson's trichrome staining of collagen fibers of Example 1, according to at least some embodiments disclosed herein.

FIG. 7A depicts a graph showing changes in plasma nitrite in peripheral blood of the groups of Example 1, according to at least some embodiments disclosed herein.

FIG. 7B depicts a graph showing changes in plasma nitrate in peripheral blood of the groups of Example 1, according to at least some embodiments disclosed herein.

FIG. 7C depicts a graph showing changes in interleukin 1 Beta in peripheral blood of the groups of Example 1, according to at least some embodiments disclosed herein.

FIG. 7D depicts a graph showing changes in interleukin 18 in peripheral blood of the groups of Example 1, according to at least some embodiments disclosed herein.

FIG. 8A depicts a graph showing mRNA expression of apoptosis genes in Example 1, according to at least some embodiments disclosed herein.

FIG. 8B depicts a graph showing caspase 3 activity for groups associated with Example 1, according to at least some embodiments disclosed herein.

### DETAILED DESCRIPTION

Unless specifically defined otherwise all technical and scientific terms used herein shall be taken to have the same meaning as commonly understood by one of ordinary skill in the art.

### Definitions

The terms “a” and “an” as used herein mean “one or more” and include the plural unless the context is inappropriate.

Reference to “about” a value or parameter herein includes (and describes) variations that are directed to that value or parameter per se. In embodiments, the term “about” refers to +/−10%, +/−5%, or +/−1%, of the designated value.

The “comprise” or variations such as “comprises” or “comprising,” will be understood to imply the inclusion of a stated element, integer or step, or group of elements, integers or steps, but not the exclusion of any other element, integer or step, or group of elements, integers or steps. Embodiments described herein also include “consisting” and/or “consisting essentially of” aspects.

“Treatment”, “treating” or similar phrases refer to obtaining beneficial or desired results, such as clinical results, for a subject, suffering from a cardiovascular disorder. As used herein, the term “treating” includes any effect, e.g., lessening, reducing, modulating, ameliorating or eliminating, that results in the improvement of the condition, disease, disorder, and the like, or ameliorating a symptom thereof. Beneficial or desired results include any one or more of: alleviating one or more symptoms of the cardiovascular disorder, diminishing the extent of the cardiovascular disorder, delaying or slowing the disease or disorder progression, and improving quality of life.

As used herein, the term “preventing” is associated with precluding a condition related to myocardial infarction and infarct expansion and/or extension following a cardiovascular disorder incident in a subject. The term “preventing” also includes precluding progression of the condition associated with the cardiovascular disorder incident in the subject.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route.

As used herein, the term “subject” refers to an organism to be treated by the methods of the present invention. Such organisms are preferably mammals (e.g., murines, simians, equines, bovines, porcines, canines, felines, and the like), and more preferably humans.

Exemplary “cardiovascular disorders” include pulmonary hypertension, systemic hypertension, angina, Cardiac syndrome X, acute myocardial infarction, stroke, aneurysm, atrial fibrillation, congestive heart failure, dilated myocardiopathy, a heart valve disease, left ventricular hypertrophy, atrial myxoma, a congenital heart disease, hypercholesterolemia, diabetes mellitus, peripheral artery disease, Raynaud's disease, pulmonary embolism, and intravascular thrombosis. In some embodiments, the cardiovascular disorder is acute myocardial infarction.

The phrase “parenteral administration” and “administered parenterally” as used herein means modes of administration other than enteral and topical administration, usually by injection, and include, without limitation, intravenous, intramuscular, intraarterial, intrathecal, intracapsular, intraorbital, intracardiac, intradermal, intraperitoneal, transtracheal, subcutaneous, subcuticular, intraarticulare, subcapsular, subarachnoid, intraspinal, and intrasternal injection and infusion.

As used herein, the term “pharmaceutically acceptable salt” refers to any circular salt (e.g., acid or base) of a compound of the present invention suitable for pharmaceutical administration which, upon administration to the subject, is capable of providing a compound of this invention or an active metabolite or residue thereof. As is known to those of skill in the art, “salts” of the compounds of the present invention may be derived from inorganic or organic acids and bases.

Examples of acids include, but are not limited to, hydrochloric, hydrobromic, sulfuric, nitric, perchloric, fumaric, maleic, phosphoric, glycolic, lactic, salicylic, succinic, toluene psulfonic, tartaric, acetic, citric, methanesulfonic, ethanesulfonic, formic, benzoic, malonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metals (e.g., sodium) hydroxides, alkaline earth metals (e.g., magnesium), hydroxides, ammonia, and compounds of formula NW⁴⁺, wherein W is C₁₋₄ alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na⁺, NH⁴⁺, and NW⁴⁺ (wherein W is a C₁₋₄ alkyl group), and the like.

For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

The terms “ABDNAZ” and “RRx-001” are used interchangeably and refer to the compound having the following structure:

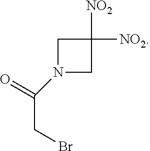 [SMILES: O=C(CBr)N1CC([N+](=O)[O-])([N+](=O)[O-])C1]

RRx-001 is associated with the chemical name 2-bromo-1-(3,3-dinitroazetidin-1-yl)ethan-1-one and is a small cyclic nitro compound that is a minimally toxic anticancer agent in Phase 3 clinical trial for the treatment of cancer. Further, RRx-001 is an electrophilic stress regulator with anti-oxidative/anti-inflammatory, vasodilatory, and cardioprotective properties. These effects are mediated by Nrf2 activation and NLRP3 inhibition, as well as nitric oxide (NO) generation under hypoxia. RRx-001 is used herein for the treatment of MI, as RRx-001 therapy alleviates MI via improved cardiac function and reversed structural remodeling in the hearts of RRx-001-treated rat subjects with MI.

Certain compounds contained in compositions of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

NLR family pyrin domain containing 3 (NLRP3) is expressed in immune cells, especially in dendritic cells and macrophages and acts as a constituent of the inflammasome. See Ghafouri-Fard (2022) Front. Immunol. 13:926895. NLRP3 acts as a pattern recognition receptor identifying pathogen-associated molecular patterns and also recognizes damage-associated molecular patterns. See Ghafouri-Fard (2022) Front. Immunol. 13:926895. Triggering of NLRP3 inflammasome by molecules ATP released from injured cells results in the activation of the inflammatory cytokines IL-1b and IL-18. Abnormal activation of NLRP3 inflammasome stimulates inflammatory or metabolic diseases. See Ghafouri-Fard (2022) Front. Immunol. 13:926895. As such, NLRP3 is a target for decreasing activity of NLRP3 inflammasome. Activation of the NLRP3 inflammasome is a primary driver of sterile inflammation in response to myocardial ischemia reperfusion. See Toldo (2019) J. Cardiovasc. Pharmacol. 73(4):215-222.

### Methods of Treatment

The present disclosure describes methods for treating or preventing a condition related to myocardial infarction and infarct expansion, extension, and/or dilatation and adverse remodeling following a cardiovascular disorder incident in a subject. The method includes administering an effective amount of RRx-001, or a pharmaceutically acceptable salt thereof, to the subject at a time period post-infarction. In some embodiments, the condition related to myocardial infarction and infarct expansion and/or extension is selected from the group consisting of: atherosclerosis, coronary artery spasm, a chronic disease, thrombosis, an embolism, and stenosis. In some embodiments, the condition related to myocardial infarction and infarct expansion and/or extension comprises the chronic disease, and the chronic disease is selected from the group consisting of: human immunodeficiency virus (HIV), diabetes, hypothyroidism, hyperthyroidism, hemochromatosis, and amyloidosis.

In some examples, the method is used for preventing cardiac remodeling after myocardial infarction, preventing cardiac dilatation after myocardial infarction, improving cardiac function after myocardial infarction, or for preventing heart failure after myocardial infarction.

In some embodiments, the effective amount of RRx-001, or a pharmaceutically acceptable salt thereof, is a therapeutically effective amount of the RRx-001, or a pharmaceutically acceptable salt thereof. Actual dosage levels of RRx-001, or a pharmaceutically acceptable salt thereof, may be varied so as to obtain an amount of the RRx-001, or a pharmaceutically acceptable salt thereof, which is effective to achieve the desired therapeutic response for a particular patient or subject, composition, and mode of administration, without being toxic to the subject.

The selected dosage level will depend upon a variety of factors including the activity of the particular compound of the present invention employed, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

In general, a suitable daily dose of a compound of the invention will be that amount of the compound which is the lowest dose effective to produce a therapeutic effect. If desired, the effective daily dose of the active compound may be administered as two, three, four, five, six or more sub-doses administered separately at appropriate intervals throughout the day, optionally, in unit dosage forms. Preferred dosing is one administration per day.

In certain embodiments, the therapeutically effective amount of RRx-001, or a pharmaceutically acceptable salt thereof, is administered at a daily dosage of from about 0.1 g/kg to about 10 mg/kg, about 1 g to about 5 mg/kg, about 0.05 mg/kg to about 10 mg/kg, about 0.1 mg/kg to about 10 mg/kg, about 0.1 mg/kg to about 5 mg/kg, about 0.5 mg/kg to about 5 mg/kg, about 0.5 mg/kg to about 3 mg/kg, about 0.1 mg/kg to about 1.5 mg/kg, about 0.1 mg/kg to about 0.35 mg/kg, about 0.35 mg/kg to about 0.75 mg/kg, or about 0.75 mg/kg to about 1 mg/kg. In preferred embodiments, the therapeutically effective amount of RRx-001, or a pharmaceutically acceptable salt thereof, is about 0.5 mg/kg.

In some embodiments, the method is used for preventing cardiac remodeling after myocardial infarction. In some embodiments, the method is used for preventing cardiac dilatation after myocardial infarction. In some embodiments, the method is used for improving cardiac function after myocardial infarction. In some embodiments, the method is used for preventing heart failure after myocardial infarction.

In some embodiments, administering a therapeutically effective amount of the RRx-001, or a pharmaceutically acceptable salt thereof, improves cardiac function in the subject experiencing at least one of systolic and/or diastolic dysfunction of the left ventricle (LV) and/or right ventricle (RV), myocardial hypertrophy, myocardial dilatation, myocardial fibrosis, depressed myocardial shortening, diminished cardiac contractility, reduced stroke volume, diminished fractional shortening, diminished ejection fraction, increased LV end diastolic pressure, increased ventricular wall stress, increased ventricular wall tension, increased LV systolic volume, increased LV diastolic volume, and/or increased thickness of the posterior wall of the heart.

In some embodiments, the therapeutically effective amount of the RRx-001, or a pharmaceutically acceptable salt thereof, results in at least one of: decreased infarct extension, decreased infarct expansion, decreased infarct scarring, reduced infarct size, increased left ventricular (LV) systolic function, and reduced myocardial hypertrophy. The cardiovascular disorder may be pulmonary hypertension, systemic hypertension, angina (e.g., Prinzmetal's angina), Cardiac syndrome X, acute MI, stroke, aneurysm, atrial fibrillation, congestive heart failure, dilated myocardiopathy, heart valve disease such as mechanical prosthetic valve, mitral rheumatic stenosis, infectious endocarditis, and marantic endocarditis, left ventricular hypertrophy, atrial myxoma and congenital heart diseases such as tetralogy of Fallot, patent foramen ovale (PFO) or atrial septal defect (ASD), atrial septal aneurysm and ventricular septal defects peripheral artery disease, Raynaud's disease, pulmonary embolism, or intravascular thrombosis. In some embodiments, the cardiovascular disorder incident is selected from the group consisting of: pulmonary hypertension, systemic hypertension, angina, Cardiac syndrome X, acute myocardial infarction, stroke, aneurysm, atrial fibrillation, congestive heart failure, dilated myocardiopathy, a heart valve disease, left ventricular hypertrophy, atrial myxoma, a congenital heart disease, hypercholesterolemia, diabetes mellitus, peripheral artery disease, Raynaud's disease, pulmonary embolism, and intravascular thrombosis.

Improvement of LV function may be measured by a variety of criteria, including measuring LV ejection fraction, which is a volume of blood pumped out of the heart during systole relative to the volume in the left ventricle at the end of diastole. LV ejection fraction may be determined by dividing a volume of blood ejected in a single ventricular contraction by end-systolic volume. It should be appreciated that FS is used to assess LV dysfunction. E′ is used to evaluate LV diastolic function based on the assumption that it reflects myocardial relaxation in the long-axis direction.

In some embodiments, the cardiac remodeling results from an acute coronary syndrome (e.g., ST elevation myocardial infarction (STEMI) or non-ST elevation acute coronary syndrome (NSTE-ACS)), cardiac fibrosis, a pulmonary embolism, pericarditis, myocarditis, coronary vasospasm, hypertension, myocardial infarction, a valvular heart disease (e.g., from a mechanical prosthetic valve, mitral rheumatic stenosis, infectious endocarditis, or marantic endocarditis), left ventricular dysfunction, cardiac ischemia, angina, tachycardia, bradycardia, hypertrophic cardiomyopathy, aortic dissection, aortitis, dilated myocardiopathy, a heart valve disease, left ventricular hypertrophy, atrial myxoma, and/or a congenital heart diseases (such as patent foramen ovale, atrial septal aneurysm and/or ventricular septal defects).

In some embodiments, administering the therapeutically effective amount of RRx-001, or a pharmaceutically acceptable salt thereof, preserves heart ejection fraction and fractional shortening and mitigates an increase in left ventricular end-systolic diameter (LVESD) and left ventricular end-diastolic diameter (LVEDD). In some embodiments, administering the therapeutically effective amount of RRx-001, or a pharmaceutically acceptable salt thereof, preserves myocardial tissue. In some embodiments, administering the therapeutically effective amount of RRx-001, or a pharmaceutically acceptable salt thereof, reduces a level of at least one serum biomarker in the subject post-infarction. In some embodiments, each serum biomarker of the at least one serum biomarker is selected from the group consisting of: lactate dehydrogenase (LDH), creatine phosphokinase-MB (CK-MB), and cardiac Troponin 1 (cTn1). In some embodiments, administering the therapeutically effective amount of RRx-001, or a pharmaceutically acceptable salt thereof, reduces at least one of: infarct expansion, infarct scarring, myocardial hypertrophy, atrial fibrosis, collagen, IL-1β level, IL-18 level, caspase 3 enzyme activity, cardiomyocytes necrosis, inflammatory cell infiltration, and fibrous hyperplasia in the subject.

### Methods for Administering RRx-001

The therapeutically effective amount of the RRx-001, or a pharmaceutically acceptable salt thereof, may be administered to the subject through a variety of methods. In some embodiments, the therapeutically effective amount of the RRx-001, or a pharmaceutically acceptable salt thereof, may be administered through a vehicle, such as dimethyl sulfoxide (DMSO).

In some embodiments, the time period post-infarction is in a range of about 1 hour to about three months. In some embodiments, the time period post-infarction is in the range of about one hour to about one month. In some embodiments, the time period post-infarction may be about 1 hour to 21 days.

In some embodiments, the effective amount of RRx-001, or a pharmaceutically acceptable salt thereof, is administered via a single administration.

In some embodiments, administering the therapeutically effective amount of RRx-001, or a pharmaceutically acceptable salt thereof, is performed via at least two administrations. In some embodiments, administering the therapeutically effective amount of RRx-001, or a pharmaceutically acceptable salt thereof, is performed at a frequency of between about 1 time per hour and about 1 time per month during the time period. In some embodiments, administering the therapeutically effective amount of RRx-001, or a pharmaceutically acceptable salt thereof, is performed at a frequency of between about 4 times per day and about 1 time per week during the time period. In some embodiments, administering the therapeutically effective amount of RRx-001, or a pharmaceutically acceptable salt thereof, is performed at a frequency of between about 2 times per day and about 3 times per week during the time period. In some embodiments, administering the therapeutically effective amount of RRx-001, or a pharmaceutically acceptable salt thereof, is performed at a frequency of about one time a day during the time period. In some embodiments, administering the therapeutically effective amount of RRx-001, or a pharmaceutically acceptable salt thereof, is performed at a frequency of between about 1 time per day and about 2 times per week during the time period.

In some embodiments, the subject is a mammal subject. In some embodiments, the mammal subject is a human subject. In some embodiments, the mammal subject is a non-human subject. In some embodiments, the treatment achieves a beneficial clinical response (e.g., the partial response or the stable disease) for at least one of the symptoms of the cardiovascular disorder.

In some embodiments, the subject is the mammal subject. In some embodiments, the subject is the human subject. In some embodiments, the subject is the non-human subject. In some embodiments, a therapeutically effective amount of RRx-001, or a pharmaceutically acceptable salt thereof, may be administered to the subject. In some embodiments, the therapeutically effective amount of RRx-001, or a pharmaceutically acceptable salt thereof, may be about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg. In some embodiments, the therapeutically effective amount of RRx-001, or a pharmaceutically acceptable salt thereof, may be formulated in a solvent. In some embodiments, the solvent may be dimethyl sulfoxide (DMSO). In some embodiments, the therapeutically effective amount of RRx-001, or a pharmaceutically acceptable salt thereof, may be administered to the subject via intravenous administration. In some embodiments, the therapeutically effective amount of RRx-001, or a pharmaceutically acceptable salt thereof, may be administered to the subject via intravenous administration during reperfusion after ischemia. In some embodiments, the therapeutically effective amount of RRx-001, or a pharmaceutically acceptable salt thereof, may be administered to the subject via intravenous administration at a time period after the reperfusion. In some embodiments, the time period is about ten minutes, about twenty minutes, about thirty minutes, about forty minutes, about fifty minutes, about an hour, about two hours, about three hours, about four hours, about five hours, about six hours, about seven hours, about eight hours, about nine hours, or about ten hours after the reperfusion. In some embodiments, echocardiography may be used to assess the effects of the therapeutically effective amount of RRx-001, or a pharmaceutically acceptable salt thereof, following MI using cardiac function parameters. In some embodiments, serum biomarkers may be measured in the subject post-infarction. In some embodiments, each serum biomarker of the at least one serum biomarker is selected from the group consisting of: lactate dehydrogenase (LDH), creatine phosphokinase-MB (CK-MB), and cardiac Troponin 1 (cTn1). In some embodiments, administering the therapeutically effective amount of RRx-001, or a pharmaceutically acceptable salt thereof, reduces at least one of: infarct expansion, infarct scarring, myocardial hypertrophy, atrial fibrosis, collagen, IL-1β level, IL-18 level, caspase 3 enzyme activity, cardiomyocytes necrosis, inflammatory cell infiltration, and fibrous hyperplasia in the subject. In some embodiments, administering the therapeutically effective amount of RRx-001, or a pharmaceutically acceptable salt thereof, after reperfused MI may decrease infarct expansion and scarring, increase LV systolic function to levels approaching normal values, and reduce myocardial hypertrophy. In some embodiments, administering the therapeutically effective amount of RRx-001, or a pharmaceutically acceptable salt thereof, after reperfused MI may reduce infarct size and/or promote administration of RRx-001 against ischemia/reperfusion injury.

### Pharmaceutical Composition

The present disclosure provides compositions or pharmaceutical compositions for preventing a condition related to MI and infarct expansion and/or extension following a cardiovascular disorder incident (e.g., post-MI) in the subject. As a general matter, the pharmaceutical composition contains at least one active agent and a pharmaceutically acceptable carrier. In some embodiments, the pharmaceutical compositions of the present invention may be specially formulated for administration in solid or liquid form, including those adapted for the following: (1) oral administration, for example, drenches (aqueous or non-aqueous solutions or suspensions), tablets (e.g., those targeted for buccal, sublingual, and/or systemic absorption), boluses, powders, granules, pastes for application to the tongue; (2) parenteral administration by, for example, subcutaneous, intramuscular, intravenous or epidural injection as, for example, a sterile solution or suspension, or sustained-release formulation; (3) topical application, for example, as a cream, ointment, or a controlled-release patch or spray applied to the skin; (4) intravaginally or intrarectally, for example, as a pessary, cream or foam; (5) sublingually; (6) ocularly; (7) transdermally; or (8) nasally.

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration. The amount of active ingredient that can be combined with a carrier material to produce a single dosage form will generally be that amount of the compound which produces a therapeutic effect. Generally, out of one hundred percent, this amount will range from about 0.1 percent to about ninety-nine percent of active ingredient, preferably from about 5 percent to about 70 percent, most preferably from about 10 percent to about 30 percent.

In certain embodiments, a formulation of the present invention comprises an excipient selected from the group consisting of cyclodextrins, celluloses, liposomes, micelle forming agents, e.g., bile acids, and polymeric carriers, e.g., polyesters and polyanhydrides; and a compound of the present invention. In certain embodiments, an aforementioned formulation renders a compound of the present invention orally bioavailable.

Methods of preparing these formulations or compositions include the step of bringing into association a compound of the present invention with the carrier and, optionally, one or more accessory ingredients. In general, the formulations are prepared by uniformly and intimately bringing into association a compound of the present invention with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules, trouches and the like), the active ingredient is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds and surfactants, such as poloxamer and sodium lauryl sulfate; (7) wetting agents, such as, for example, cetyl alcohol, glycerol monostearate, and non-ionic surfactants; (8) absorbents, such as kaolin and bentonite clay; (9) lubricants, such as talc, calcium stearate, magnesium stearate, solid polyethylene glycols, sodium lauryl sulfate, zinc stearate, sodium stearate, stearic acid, and mixtures thereof; (10) coloring agents; and (11) controlled release agents such as crospovidone or ethyl cellulose. In the case of capsules, tablets and pills, the pharmaceutical compositions may also comprise buffering agents. Solid compositions of a similar type may also be employed as fillers in soft and hard-shelled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be formulated for rapid release, e.g., freeze-dried. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically-acceptable carrier, and with any preservatives, buffers, or propellants which may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

In certain embodiments, it may be desirable to introduce the compositions disclosed herein into the central nervous system by any suitable route, including intraventricular, intrathecal and epidural injection. Intraventricular injection may be facilitated by an intraventricular catheter, for example, attached to a reservoir, such as an Ommaya reservoir.

In some embodiments, the pharmaceutical composition is configured as an inhalable formulation. In some embodiments, the inhalable formulation is configured as a dosage form adapted for pulmonary or nasal administration to the subject. In some embodiments, for example, dosage forms may include those adapted for inhalation such as aerosols and dry powders. In some embodiments, the formulation described herein is suitable for topical delivery to the lung via nose inhalation and/or mouth inhalation. In other embodiments, the compositions disclosed herein may also be administered directly to the lung by inhalation by a number of different devices.

In some embodiments, the inhalable formulation is configured as an aerosol formulation that comprises a propellant. In some embodiments, the propellant can provide energy to deliver molecules of any of the compounds described herein to the lung. Representative propellants are disclosed in U.S. Pat. No. 6,932,962 B1 and U.S. Pat. No. 8,367,734 B1. In some embodiments, the propellant is presented in the aerosol formulation in an amount ranging from 98% to 99% (w/w) relative to the total weight of the aerosol formulation.

In some embodiments, the aerosol formulation further comprises a surfactant, a co-solvent, and/or a pH buffer. The surfactant can give fine dispersions of the compounds described herein in the propellant and can stabilize the mixture of the compounds described herein in the propellant. In some embodiments, the surfactant comprises a fatty acid or a pharmaceutically acceptable salt thereof (e.g., a therapeutically effective amount), a bile salt, a phospholipid, or an alkyl saccharide. In some embodiments, the surfactant is presented in the formulations described herein in an amount of less than 5% (w/w) (e.g., less than 4%, less than 3%, less than 2%, less than 1% by weight) relative to the total weight of the aerosol formulation.

In some embodiments, the co-solvent can help to stabilize the surfactant and improve the dispersion characteristics. In some embodiments, exemplary co-solvents include ethyl alcohol, isopropyl alcohol, propylene glycol, ethylene glycol, propane, butane, isobutane, pentane, dimethyl ether, diethyl ether and the like. In some embodiments, the co-solvent is present in the formulation in an amount ranging from 0.5% to 20% w/w of the total weight of the formulation. In some embodiments, the co-solvent is present in the formulation in an amount ranging from 0.5% to 5% w/w of the total weight of the formulation. In some embodiments, the co-solvent is present in the formulation in an amount ranging from 0.5% to 1.5% (w/w) of the total weight of the formulation. Representative surfactants, co-solvents, and pH buffers are disclosed in U.S. Pat. No. 6,932,962 B1 and U.S. Pat. No. 8,367,734 B1.

In some embodiments, provided herein are combinations containing the aerosol formulation with the propellant and a pressurized bottle or a nebulizer. In some embodiments, the aerosol formulation with the propellant may be packed in pressurized bottles, where a dosage controller may be used with the pressurized bottle to control the amount of drug being administrated in each spray. In some embodiments, the aerosol formulation with the propellant may be packed in pressurized bottles with a dosage controller, where the dosage controller comprises a valve that controls the delivery of a metered amount of the drug.

In some embodiments, the aerosol formulation is propellant-free and comprises the effective amount of the RRx-001 or the pharmaceutical composition and a solvent. In some embodiments, exemplary solvents include water and alcohols, such as ethanol, isopropanol, and glycols, such as propylene glycol, polyethylene glycol, polypropylene glycol, glycol ether, glycerol and polyoxyethylene alcohols. In some embodiments, the solvent is present in the propellant-free aerosol formulation in an amount ranging from about 0.01% to about 90% (w/w), or about 0.01% to about 50% (w/w), or about 0.01% to about 25% (w/w), or about 0.01% to about 10% (w/w), or about 0.01% to about 5% (w/w) relative to the total weight of the aerosol formulation.

In some embodiments, the propellant-free aerosol formulation may further comprise an emulsifying agent. In some embodiments, exemplary emulsifying agents are disclosed in U.S. Pat. No. 9,498,437 B2. In some embodiments, the emulsifying agent is present in the propellant-free aerosol formulations in an amount ranging from about 0.001% to about 50% (w/w), or about 0.001% to about 25% (w/w), or about 0.001% to about 10% (w/w), or about 0.001% to about 2% (w/w), or about 0.001% to about 1% (w/w) relative to the total weight of the aerosol formulation.

In some embodiments, the propellant-free aerosol formulation may further comprise a complexing agent. In some embodiments, exemplary complexing agents include, but are not limited to, ethylenediaminetetraacetic acid (EDTA) or a pharmaceutically acceptable salt thereof (e.g., a therapeutically effective amount), such as the disodium salt, citric acid, nitrilotriacetic acid and the salts thereof, and sodium edetate. Representative complexing agents are disclosed in U.S. Pat. No. 9,498,437 B2. In some embodiments, the complexing agent is present in the propellant-free aerosol formulations in an amount ranging from about 0.001% to about 50% (w/w), or about 0.001% to about 25% (w/w), or about 0.001% to about 10% (w/w), or about 0.001% to about 2% (w/w), or about 0.001% to about 1% (w/w) relative to the total weight of the aerosol formulation.

In some embodiments, the propellant-free aerosol formulation may further comprise a tonicity agent that can adjust the isotonicity of the present formulations. In some embodiments, exemplary tonicity agents include, but are not limited to, sodium chloride, potassium chloride, zinc chloride, calcium chloride or mixtures thereof. Other osmotic adjusting agents may also include, but are not limited to, mannitol, glycerol, and dextrose or mixtures thereof.

Representative tonicity agents are disclosed in U.S. Pat. No. 9,498,437 B2. In some embodiments, the tonicity agent is present in the propellant-free aerosol formulations in an amount ranging from about 0.01% to about 10% (w/w), or about 1% to about 10% (w/w), or about 1% to about 6% (w/w) relative to the total weight of the aerosol formulation. In some embodiments, the aerosol formulation may further comprise the pH buffer.

In some embodiments, provided herein are combinations containing the propellent-free aerosol formulation provided herein and a nebulizer. In some embodiments, the nebulizer can nebulize liquid formulations, including the propellant-free aerosol formulations detailed herein, and produce a nebulized aerosol mist. In some embodiments, the nebulizer may further have an internal baffle, which can selectively remove large droplets from the mist by impaction and allow the droplets to return to the reservoir, so that only fine aerosol droplets are entrained into the lung of the subject by the inhaling air/oxygen. Examples of nebulizers include devices supplied by Sheffield Pharmaceuticals, St. Louis, MO. (Armer et al., U.S. Pat. No. 5,954,047; van der Linden et al., U.S. Pat. No. 5,950,619; van der Linden et al., U.S. Pat. No. 5,970,974) and Batelle Pulmonary Therapeutics, Columbus, OH).

In some embodiments, a Metered Dose Inhaler (“MDI”), which utilizes canisters that contain a suitable low boiling propellant, (e.g., dichlorodifluoromethane, trichlorofluoromethane, dichlorotetrafluoroethane, carbon dioxide or any other suitable gas) may be used to deliver the RRx-001 and/or pharmaceutical compositions thereof directly to the lung. Specifically, the MDI comprises an aerosol container suitable for containing a propellant-based aerosol formulation and/or a metering valve, for example a side valve, which controls the release of the aerosol formulation to the subject. Representative methods and devices to administer the aerosol formulation with the propellant are disclosed in U.S. Pat. No. 9,498,437 B2.

In another embodiment, a Dry Powder Inhaler (“DPI”) device may be used to administer the compositions disclosed herein to the lung. DPI devices typically use a mechanism such as a burst of gas to create a cloud of dry powder inside a container, which may then be inhaled by the patient and are well known in the art. In a particular embodiment, a popular variation is the multiple dose DPI (“MDDPI”) system, which allows for the delivery of more than one therapeutic dose. MDDPI devices are commercially available from a number of pharmaceutical companies e.g., Schering Plough, Madison, NJ). For example, capsules and cartridges of gelatin for use in an inhaler or insufflator may be formulated containing a powder mix of the compositions disclosed herein and a suitable powder base such as lactose or starch for these systems.

In some embodiments, another type of device that may be used to deliver the compositions disclosed herein to the lung is a liquid spray device supplied, for example, by Aradigm Corporation, Hayward, CA. Liquid spray systems use extremely small nozzle holes to aerosolize liquid drug formulations that may then be directly inhaled into the lung.

In some embodiments, a nebulizer is used to deliver the compositions disclosed herein to the lung. Nebulizers create aerosols from liquid drug formulations by using, for example, ultrasonic energy to form fine particles that may be readily inhaled (see e.g., Verschoyle et al., British J. Cancer, 1999, 80, Suppl. 2, 96). Examples of nebulizers include devices supplied by Sheffield Pharmaceuticals, St. Louis, MO. (Armer et al., U.S. Pat. No. 5,954,047; van der Linden et al., U.S. Pat. No. 5,950,619; van der Linden et al., U.S. Pat. No. 5,970,974) and Batelle Pulmonary Therapeutics, Columbus, OH).

In other embodiments, an electrohydrodynamic (“EHD”) aerosol device is used to deliver the compositions disclosed herein to the lung of a patient. EHD aerosol devices use electrical energy to aerosolize liquid drug solutions or suspensions (see e.g., Noakes et al., U.S. Pat. No. 4,765,539). The electrochemical properties of the formulation may be important parameters to optimize when delivering the RRx-001 and/or pharmaceutical composition thereof to the lung with an EHD aerosol device. EHD aerosol devices may more efficiently deliver drugs to the lung than existing pulmonary delivery technologies.

Suitable pharmaceutically acceptable excipients will vary depending upon the particular dosage form chosen. In some embodiments, for example, certain pharmaceutically acceptable excipients may be chosen for their ability to: facilitate the production of aerosol for inhalation, facilitate the production of solution or mist for inhalation, facilitate the production of dry powder for inhalation, or facilitate the production of stable dosage forms.

In some embodiments, the compositions disclosed herein can be delivered via sustained release systems, e.g., oral sustained release systems. In other embodiments, a pump may be used (e.g., Langer, supra, Sefton, 1987, CRC Crit. Ref Biomed. Eng. 14:201; Saudek et al., 1989, N. Engl. J Med. 321:574).

In some embodiments, polymeric materials can be used (e.g., “Medical Applications of Controlled Release,” Langer and Wise (eds.), CRC Press, Boca Raton, Florida (1974); “Controlled Drug Bioavailability,” Drug Product Design and Performance, Smolen and Ball (eds.), Wiley, New York (1984); Ranger et al., 1983, J Macromol. Sci. Rev. Macromol Chem. 23:61; Levy et al., 1985, Science 228: 190; During et al., 1989, Ann. Neurol. 25:351; Howard et al., 1989, J. Neurosurg. 71:105).

In other embodiments, polymeric materials are used for oral sustained release delivery. Polymers include, but are not limited to, sodium carboxymethylcellulose, hydroxypropylcellulose, hydroxypropylmethylcellulose and hydroxyethylcellulose (most preferred, hydroxypropyl methylcellulose). Other cellulose ethers have been described (Alderman, Int. J. Pharm. Tech. & Prod. Mfr. 1984, 5(3) 1-9). Factors affecting drug release are well known to the skilled artisan and have been described in the art (Bamba et al., Int. J. Pharm. 1979, 2, 307).

In other embodiments, enteric-coated preparations can be used for oral sustained release administration. Coating materials include polymers with a pH-dependent solubility (i.e., pH-controlled release), polymers with a slow or pH-dependent rate of swelling, dissolution or erosion (i.e., time-controlled release), polymers that are degraded by enzymes (i.e., enzyme-controlled release) and polymers that form firm layers that are destroyed by an increase in pressure (i.e., pressure-controlled release).

In other embodiments, osmotic delivery systems are used for oral sustained release administration (Verma et al., Drug Dev. Ind. Pharm., 2000, 26:695-708). In some embodiments, OROS™ osmotic devices are used for oral sustained release delivery devices (Theeuwes et al., U.S. Pat. No. 3,845,770; Theeuwes et al., U.S. Pat. No. 3,916,899).

In yet other embodiments, a controlled-release system can be placed in proximity of the target of RRx-001 described herein and/or pharmaceutical composition, thus requiring only a fraction of the systemic dose (e.g., Goodson, in “Medical Applications of Controlled Release,” supra, vol. 2, pp. 115-138 (1984)). Other controlled-release systems previously may also be used (Langer, 1990, Science 249:1527-1533).

Pharmaceutical compositions of this invention suitable for parenteral administration comprise one or more compounds of the invention in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain sugars, alcohols, antioxidants, buffers, bacteriostats, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms upon the subject compounds may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a drug, it is desirable to slow the absorption of the drug administered by subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered drug form is accomplished by dissolving or suspending the drug in an oil vehicle.

Injectable depot forms are made by forming microencapsule matrices of the subject compounds in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissue.

When the compounds of the present invention are administered as pharmaceuticals, to human and animal subjects, they can be given per se or as a pharmaceutical composition containing, for example, 0.1 to 99% (more preferably, 10 to 30%) of active ingredient in combination with a pharmaceutically acceptable carrier.

The preparations of the present invention may be given orally, parenterally, topically, or rectally. They are of course given in forms suitable for each administration route. For example, they are administered in tablets or capsule form, by injection, inhalation, eye lotion, ointment, suppository, etc. administration by injection, infusion or inhalation; topical by lotion or ointment; and rectal by suppositories. Parenteral administrations are preferred.

Combination with a Blood Product and/or Another Agent

The pharmaceutical composition comprises the effective amount of RRx-001, or a pharmaceutically acceptable salt thereof (e.g., a therapeutically effective amount), and at least one pharmaceutically acceptable carrier or excipient. In some embodiments, the pharmaceutical composition comprises (1) an effective amount of RRx-001, or a pharmaceutically acceptable salt thereof and (2) at least one of a blood product and an additional agent. In various embodiments disclosed herein, the composition comprising the effective amount of RRx-001, or a pharmaceutically acceptable salt thereof, is combined with a blood product prior to administration to the subject. It should be appreciated that the blood product can be blood harvested from the subject or may be harvested from a donor that matches the blood type of the subject.

In some embodiments, the blood product comprises erythrocyte cells. In some embodiments, the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis. In some embodiments, the blood product is a mixture of packed red blood cells. In other embodiments, the blood product is whole blood. In some embodiments, the whole blood is autologous whole blood. In some embodiments, the whole blood is donor-matched allogenic whole blood.

Each dose of the composition comprising the effective amount of RRx-001, or a pharmaceutically acceptable salt thereof, can be administered to the subject by intravenous infusion. Depending on the circumstances, the composition is pre-mixed with a sample of blood harvested from the subject prior to administration. Methods of performing blood transfusion by administering to a subject in need thereof are described in WO2017/123593, the content of which is incorporated herein by reference.

In some embodiments, administering the composition comprising the effective amount of RRx-001, or a pharmaceutically acceptable salt thereof, does not lead to toxic systemic side effects. In some embodiments, the toxic systemic side effects are selected from the group consisting of dyspnea, cough, fatigue, cardiac symptoms, electrolyte deficiencies, thyroid dysfunction, bone loss, sleep issues, nephrotoxicity, neurologic symptoms, autoimmunity, retinitis, hepatotoxicity, gastrointestinal distress, weight loss, malaise, rash, and leukopenia with increased risk of infection and the development of malignancy, and combinations thereof.

In some embodiments, the method further comprises administering an agent prior to, concurrently with, or subsequent administering the effective amount of RRx-001, or a pharmaceutically acceptable salt thereof. In some embodiments, the agent is selected from the group consisting of: a statin, an angiotensin-converting-enzyme (ACE) inhibitor, aspirin, oxygen, a nitric oxide donor, a drug-eluting stent, an anticoagulant or antiplatelet therapy, a phosphodiesterase-5 inhibitor, a calcium channel blocker, an angiotensin II receptor blocker (ARB), a beta blocker, an aldosterone antagonist, a loop diuretic, an epigenetic inhibitor, and a nitrite.

In some embodiments, the effective amount of RRx-001, or a pharmaceutically acceptable salt thereof, is combined with a statin. In some embodiments, the effective amount of RRx-001, or a pharmaceutically acceptable salt thereof, is combined with an ACE inhibitor. In some embodiments, the effective amount of RRx-001, or a pharmaceutically acceptable salt thereof, is combined with an ARB. In some embodiments, the effective amount of RRx-001, or a pharmaceutically acceptable salt thereof, is combined with a beta blocker. In some embodiments, the effective amount of RRx-001, or a pharmaceutically acceptable salt thereof, is combined with an aldosterone antagonist. In some embodiments, the effective amount of RRx-001, or a pharmaceutically acceptable salt thereof, is combined with a loop diuretic. In some embodiments, the effective amount of RRx-001, or a pharmaceutically acceptable salt thereof, is combined with an epigenetic inhibitor. In some embodiments, the effective amount of RRx-001, or a pharmaceutically acceptable salt thereof, is combined with nitrite. In some embodiments, an amount of the agent is in a range of about 1 mg to about 50 mg. In some embodiments, the amount of the agent is in the range of about 1 mg to about 15 mg.

In some embodiments, administering the agent prior to, concurrently with, or subsequent administering the effective amount of RRx-001, or a pharmaceutically acceptable salt thereof, prevents at least one side effect associated with the agent. In some embodiments, administering the agent prior to, concurrently with, or subsequent administering the effective amount of RRx-001, or a pharmaceutically acceptable salt thereof, reduces at least one side effect associated with the agent.

The present invention can provide methods of attenuating interactions of a first drug (e.g., a first therapeutic agent) and a second drug (e.g., a second therapeutic agent) in a mammal. As described herein, interactions of drugs, or drug-drug interactions, can refer to the changes of the effects of a drug or a pharmaceutical composition on a mammal when the pharmaceutical composition is taken together with a second drug or second pharmaceutical composition. In some embodiments, the interactions can occur when more than two drugs are concurrently in a mammal, regardless of the time between the administrations of the two or more drugs and thereby, and react with each other.

In some embodiments, an attenuating interaction of drugs includes actions that result in reducing or preventing any types of interactions between two or more drugs or reducing the hypersensitivity, the toxicity, or adverse effects that are caused by the interactions of two or more drugs. In some embodiments, the interactions can include, but are not limited to, synergistic or antagonistic interactions. By way of examples, attenuating interactions of the drugs can be at least any one of the following scenarios: reducing and/or preventing drug-drug physical interactions, reducing and/or preventing drug-drug pharmacokinetic interactions, reducing and/or preventing the hypersensitivity caused by co-existence of the drugs, reducing and/or preventing the toxicity caused by co-existence of drugs, or reducing and/or preventing the antagonistic interactions of drugs.

In some embodiments, the effects of the attenuated interactions can be delayed, decreased, or enhanced absorption of either pharmaceutical composition, and thereby decreases or increases the action of one or more of the additional agent(s) or the pharmaceutical composition. In some embodiments, the attenuated interactions can impact the transport or the distribution of the additional agent(s) or the pharmaceutical compositions.

Accordingly, in certain embodiments, the subject has reduced incidence and/or severity of side effects compared to subjects receiving a direct administration of the same additional agent(s) at the same dose without being mixed with the blood product prior to administration. In certain embodiments, the subject has reduced side effects compared to subjects receiving a direct administration of the same additional agent(s) at the same dose without being mixed with the blood product prior to administration. In certain embodiments, the dose of the additional agent(s) in the pharmaceutical composition is at least about 10% to about 300% more than the dose recommended for a direct administration of the same additional agent(s) without being mixed with the blood product prior to administration. In certain embodiments, the dose of the additional agent(s) in the pharmaceutical composition is at least 1%, at least 5%, at least 10%, at least 20%, at least 30%, at least 40%, at least 50%, at least 60%, at least 70%, at least 80%, at least 90%, at least 100%, at least 200%, at least 300%, at least 400%, at least 500%, at least 600%, at least 700%, at least 800%, at least 900%, at least 1000%, or higher, inclusive of all ranges and subranges therebetween, more than the dose recommended for a direct administration of the same additional agent(s) without being mixed with the blood product prior to administration.

In certain embodiments, the additional agent(s) has/have a longer circulating half-life in the subject compared to direct administration of the same additional agent(s) at the same dose without being mixed with the blood product prior to administration. In certain embodiments, the circulating half-life of the additional agent(s) is at least about 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 100%, 110%, 110%, 120%, 130%, 140%, 150%, 160%, 170%, 180%, 190%, 200%, 250%, 300%, 350%, 400%, 450%, 500%, 600%, 700%, 800%, 900%, 1000%, or more, longer than the circulating half-life of the same additional agent(s) at the same dose without being mixed with the blood product before administration.

### Amount of RRx-001

The therapeutically effective amount of the RRx-001, or the pharmaceutically acceptable salt thereof, may be administered to the subject in various dosages at various frequencies and over various periods of time. Each of the dosage, frequency of the dosage, and the periods of time to which the dosage is administered may be adjusted based on factors, including, but not limited, to the medical condition of the subject, the desired outcome, and the one or more (if any) agents that are administered concurrently with the therapeutically effective amount of the RRx-001, or the pharmaceutically acceptable salt thereof.

In various embodiments, the dosage may be measured in units of a mass of the RRx-001 or the pharmaceutically acceptable salt thereof, a volume of the RRx-001 or the pharmaceutically acceptable salt thereof, a mass of the RRx-001 or the pharmaceutically acceptable salt thereof to a mass of the subject, a mass of the RRx-001 or the pharmaceutically acceptable salt thereof to a volume of the subject, a volume of the RRx-001 or the pharmaceutically acceptable salt thereof to a volume of the subject, a mass of the RRx-001 or the pharmaceutically acceptable salt thereof to a surface area of the subject, a volume of the RRx-001 or the pharmaceutically acceptable salt thereof to a surface area of the subject, or the like.

In some embodiments, the therapeutically effective amount of the RRx-001, or the pharmaceutically acceptable salt thereof, may be administered in one or more doses in a range of about 0.1 mg and about 500.0 mg. In some embodiments, the therapeutically effective amount of the RRx-001, or the pharmaceutically acceptable salt thereof, may be administered in one or more doses in a range of about 0.1 mg and about 200.0 mg. In some embodiments, the therapeutically effective amount of the RRx-001, or the pharmaceutically acceptable salt thereof, may be administered in one or more doses in a range of about 0.1 mg and about 0.5 mg, about 0.5 mg and about 1.0 mg, about 1.0 mg and about 1.5 mg, about 1.5 mg and about 2.0 mg, about 2.0 mg and about 2.5 mg, about 2.5 mg and about 3.0 mg, about 3.0 mg and about 3.5 mg, about 3.5 mg and about 4.0 mg, about 4.0 mg and about 4.5 mg, about 4.5 mg and about 5.0 mg, about 5.0 mg and about 6.0 mg, about 6.0 mg and about 7.0 mg, about 7.0 mg and about 8.0 mg, about 8.0 mg and about 9.0 mg, about 9.0 mg and about 10.0 mg, about 10.0 mg and about 15.0 mg, about 15.0 mg and about 20.0 mg, about 20.0 mg and about 25.0 mg, about 25.0 mg and about 30.0 mg, about 30.0 mg and about 35.0 mg, about 35.0 mg and about 40.0 mg, about 40.0 mg and about 45.0 mg, about 45.0 mg and about 50.0 mg, about 50.0 mg and about 55.0 mg, about 55.0 mg and about 60.0 mg, about 60.0 mg and about 65.0 mg, about 65.0 mg and about 70.0 mg, about 70.0 mg and about 75.0 mg, about 75.0 mg and about 80.0 mg, about 80.0 mg and about 85.0 mg, about 85.0 mg and about 90.0 mg, about 90.0 mg and about 95.0 mg, about 95.0 mg and about 100.0 mg, about 100.0 mg and about 110.0 mg, about 110.0 mg and about 120.0 mg, about 120.0 mg and about 130.0 mg, about 130.0 mg and about 140.0 mg, about 140.0 mg and about 150.0 mg, about 150.0 mg and about 160.0 mg, about 160.0 mg and about 170.0 mg, about 170.0 mg and about 180.0 mg, about 180.0 mg and about 190.0 mg, or about 190.0 mg and about 200.0 mg. In other embodiments, the therapeutically effective amount of the RRx-001, or the pharmaceutically acceptable salt thereof, may be administered in one or more doses in a range of about 100.0 mg and about 120.0 mg, about 120.0 mg and about 140.0 mg, about 140.0 mg and about 160.0 mg, about 160.0 mg and about 180.0 mg, about 200.0 mg and about 220.0 mg, about 220.0 mg and about 240.0 mg, about 240.0 mg and about 260.0 mg, about 260.0 mg and about 280.0 mg, about 280.0 mg and about 300.0 mg, about 300.0 mg and about 320.0 mg, about 320.0 mg and about 340.0 mg, about 340.0 mg and about 360.0 mg, about 360.0 mg and about 380.0 mg, about 380.0 mg and about 400.0 mg, about 400.0 mg and about 420.0 mg, about 420.0 mg and about 440.0 mg, about 440.0 mg and about 460.0 mg, about 460.0 mg and about 480.0 mg, or about 480.0 mg and about 500.0 mg.

In some embodiments, where the subject is a human subject, the therapeutically effective amount of the RRx-001, or the pharmaceutically acceptable salt thereof is in a range of from about 0.1 mg to about 500.0 mg, and more preferably, in the range from about 0.5 mg to about 200.0 mg. In other embodiments, where the subject is a non-human subject, the therapeutically effective amount of the RRx-001, or the pharmaceutically acceptable salt thereof is in a range of from about 0.1 mg/kg of the non-human subject to about 100.0 mg/kg of the non-human subject, and more preferably, in the range from about 1 mg/kg of the non-human subject to about 50.0 mg/kg of the non-human subject.

### Frequency of Administration

In some embodiments, the therapeutically effective amount of the RRx-001, or the pharmaceutically acceptable salt thereof may be administered via a single administration. In some embodiments, the therapeutically effective amount of the RRx-001, or the pharmaceutically acceptable salt thereof, may be administered via at least two administrations. In other embodiments, each administration dosage of the therapeutically effective amount of the RRx-001, or the pharmaceutically acceptable salt thereof may be administered at various intervals or frequencies over a time period until the end of a dosing period.

In some embodiments, each dose of the therapeutically effective amount of the RRx-001, or the pharmaceutically acceptable salt thereof, is administered at a frequency of about 1 dose per 24 hours. In another embodiment, each dose of the therapeutically effective amount of the RRx-001, or the pharmaceutically acceptable salt thereof, is administered at a frequency of about 1 dose per 48 hours. In some embodiments, each dose of the therapeutically effective amount of the RRx-001, or the pharmaceutically acceptable salt thereof, is administered at a frequency of about 3 doses per week. In another embodiment, each dose of the therapeutically effective amount of the RRx-001, or the pharmaceutically acceptable salt thereof, is administered at a frequency of about 2 doses per week. In some embodiments, each dose of the therapeutically effective amount of the RRx-001, or the pharmaceutically acceptable salt thereof, is administered at a frequency of about 1 dose per week. In other embodiments, each dose of the therapeutically effective amount of the RRx-001, or the pharmaceutically acceptable salt thereof, is administered at a frequency of about 2 doses per month. In some embodiments, each dose of the therapeutically effective amount of the RRx-001, or the pharmaceutically acceptable salt thereof, is administered at a frequency of about 1 dose per month. In other embodiments, each dose of the therapeutically effective amount of the RRx-001, or the pharmaceutically acceptable salt thereof, is administered at a frequency of about 1 dose per 2 months. In some embodiments, each dose of the therapeutically effective amount of the RRx-001, or the pharmaceutically acceptable salt thereof, is administered at a frequency of about 1 dose per 3 months.

In other embodiments, each dose of the therapeutically effective amount of the RRx-001, or the pharmaceutically acceptable salt thereof, is administered at a frequency of between about 1 dose per day and about 1 dose per week until the end of a dosing period. In some embodiments, each dose of the therapeutically effective amount of the RRx-001, or the pharmaceutically acceptable salt thereof, is administered at a frequency of between about 1 dose per day and about 2 doses per week until the end of a dosing period. In other embodiments, each dose of the therapeutically effective amount of the RRx-001, or the pharmaceutically acceptable salt thereof, is administered at a frequency of between about 1 dose per week and about 1 dose per month until the end of a dosing period. In other embodiments, each dose of the therapeutically effective amount of the RRx-001, or the pharmaceutically acceptable salt thereof, is administered at a frequency of between about 1 dose per month and about 1 dose per 3 months until the end of a dosing period.

In some embodiments, the therapeutically effective amount of the RRx-001, or the pharmaceutically acceptable salt thereof, may be administered at a frequency of once per day, once per week, once per month, etc. during a time period.

### EXAMPLE

In order that this disclosure may be more fully understood, the following Example is set forth. It should be understood that this example is for illustrative purposes only and is not to be construed as limiting this disclosure in any manner.

### Example 1. Assessing the Efficacy of RRx-001 as a Treatment after Reperfused Acute MI

Animal experiments were performed in accordance with protocols approved. Male Wistar rats or subjects (about 8-9 weeks old) were purchased from Charles River (San Diego, California) and allowed about 1 week to acclimatize to the new environment prior to random allocation into four groups (n=8 per group). The groups included: a first group 106 (sham), a second group 108 (RRx-001), a third group 110 (RRx-001 I/R), and a fourth group 112 (I/R).

The first group 106 included sham subjects (e.g., no treatment). The subjects of the second group 108 were given about 5 mg/kg of RRx-001 prepared in dimethyl sulfoxide (DMSO) and administered to the subjects via intravenous administration. The subjects of the second group 108 were not subjected to ischemia. The subjects of the third group 110 were administered about 0.5 mg/kg of RRx-001 prepared in DMSO via intravenous administration during reperfusion after ischemia. The subjects of the fourth group 112 were untreated, but subjected to ischemia and reperfusion. A subset of the patients (n=8) per group were studied at Day 2, and the remaining subjects (n=4) were studied at Day 7.

Regarding the myocardial ischemia reperfusion, the subjects were anesthetized with isoflurane (5% induction and 2% maintenance in 30% oxygen) and placed on a thermal heating pad. Subject ventilation was supported using a rodent ventilator (60 strokes/minute with tidal volume of 8 mL/kg) via an endotracheal tube (PE-50). The pericardium of the subjects was exposed and detached via incision between the third and fourth intercostal ribs, a silk suture (0-6) was passed under the left anterior descending artery (LAD), and a slipknot was made to occlude flow and create ischemia for a time period of about 30 minutes. The occlusion was confirmed by blanching of the tissue distal to the ligation site. The knot was removed, the chest was closed, and the hearts of the subjects were monitored for a time period of one hour during reperfusion. An effective amount of RRx-001 (e.g., about 5 mg/kg) or a vehicle was given at 1 hour after reperfusion.

The structural and functional cardiac remodeling, infarct expansion and fibrosis, as well as cardiomyocyte hypertrophy and the expression of inflammatory genes were assessed. Myocardial injury was evaluated by measuring the activity of lactate dehydrogenase (LDH), creatine kinase (CK) enzymes in both plasma and tissue following the standard protocol, described by the Sigma-Aldrich kit (MAK066 and MAK116).

Statistical analysis was also performed using GraphPad Prism 9 (GraphPad Software, Inc.). Results were expressed as the mean+standard deviation. One-way ANOVA was performed to compare between groups. Post hoc Bonferroni tests were carried out to compare individual mean values when significant differences were found. Differences were considered significant at P<0.05.

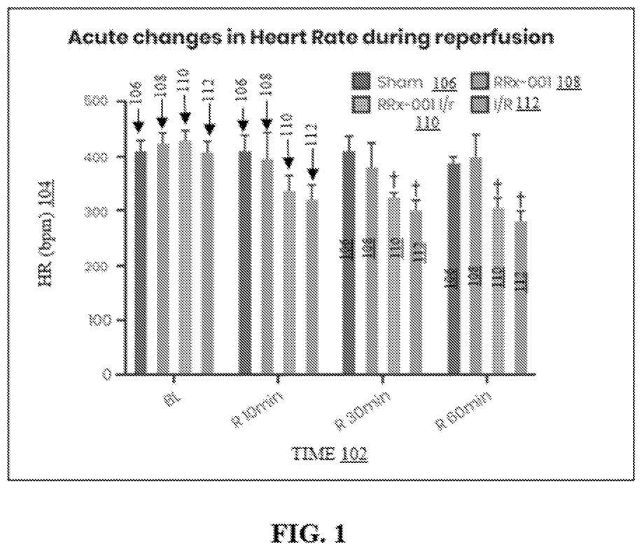

FIG. 1 depicts a graph comparing acute changes in heart rate (HR) during reperfusion for various groups of Example 1, according to at least some embodiments disclosed herein. FIG. 1 includes an x-axis 102 associated with time (measured in minutes) and a y-axis 104 associated with HR (measured in beats per minute or “bpm”). As shown in FIG. 1, infusion of the RRx-001 did not result in any acute changes in HR in sham animals (e.g., the first group 106), or relative to animals with I/R (e.g., the fourth group 112).

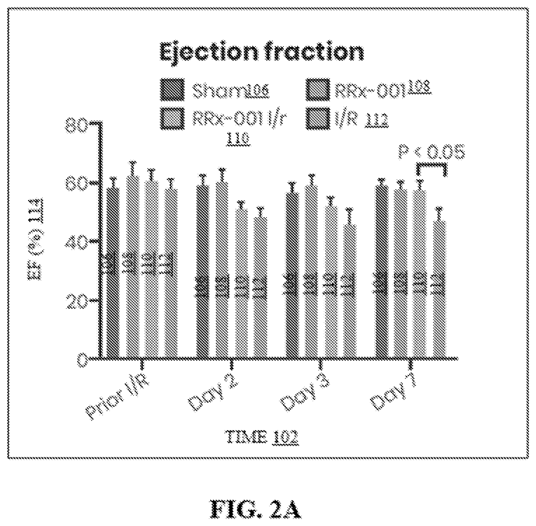

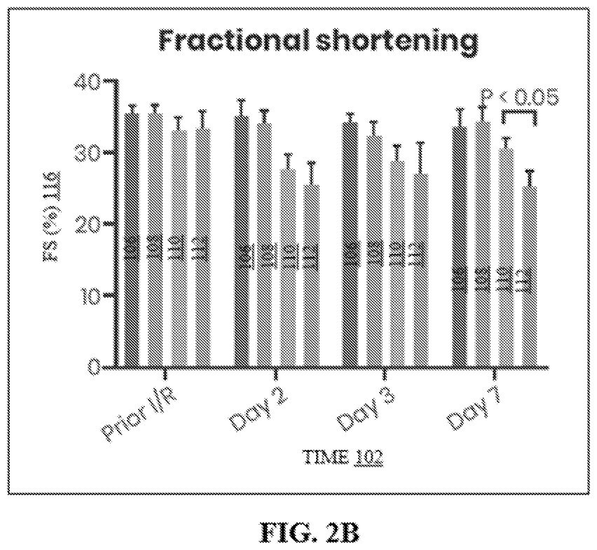

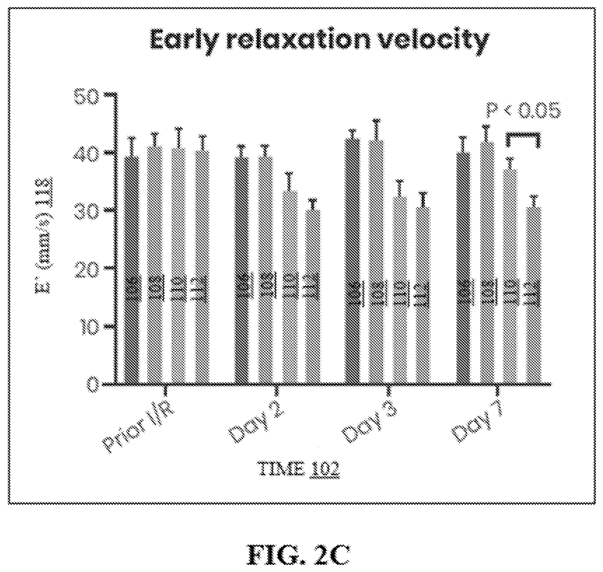

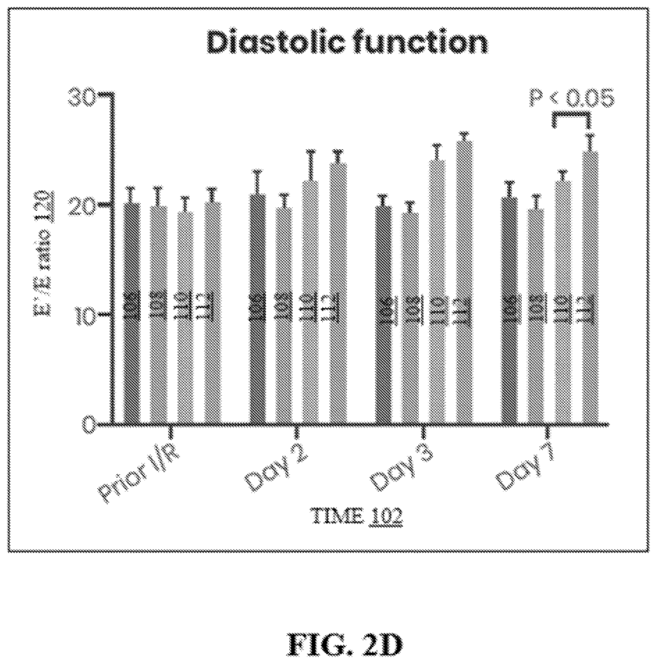

FIGS. 2A-2D depict echocardiographic results. Echocardiography was used to assess the protective effects of RRx-001 treatment following MI using various cardiac function parameters. For the heart ejection fraction (or EF, which is a percentage of blood leaving a heart of the subject each time it contracts), the subjects of the first group 106, the second group 108, the third group 110, and the fourth group 112 were anesthetized (1% isoflurane) and echocardiographic measurements were conducted using a VisualSonics Vevo770 echocardiogram device with a 30-MHz linear transducer. M-mode recordings from the short axis was used to determine EF and FS.

Specifically, FIG. 2A depicts a graph showing heart EF amounts for various groups of Example 1, according to at least some embodiments disclosed herein. FIG. 2A includes the x-axis 102 associated with time (measured prior to I/R and in days subsequent I/R) and a y-axis 114 (associated with EF, measured as a percentage). FIG. 2B depicts a graph showing fractional shortening (or FS) amounts for various groups of Example 1, according to at least some embodiments disclosed herein. FIG. 2B includes the x-axis 102 associated with time (measured prior to I/R and in days subsequent I/R) and a y-axis 116 (associated with FS, measured as a percentage). As seen in both FIG. 2A and FIG. 2B, EF and FS were preserved in subjects treated with RRx-001 and subjected to I/R (e.g., the third group 110). In case of the subjects that underwent I/R (e.g., the fourth group 112), the left ventricular end-systolic diameter (LVESD) and left ventricular end-diastolic diameter (LVEDD) increased compared to sham (e.g., the first group 106), and treatment with RRx-001 mitigated the increase, but did not prevent their increase.

FIG. 2C depicts early relaxation velocity (or E′) for various groups of Example 1 and FIG. 2D depicts a graph showing diastolic function (or an E′/E ratio) for various groups of Example 1, according to at least some embodiments disclosed herein. FIG. 2C has the x-axis 102 associated with time (measured prior to I/R and in days subsequent I/R) and a y-axis 118 associated with E′ (measured in mm/s). FIG. 2D has the x-axis 102 associated with time (measured prior to I/R and in days subsequent I/R) and a y-axis 120 associated with the E′/E ratio. As shown in FIG. 2C and FIG. 2D, cardiac function as measured by echocardiography showed the positive effects of RRx-001 on the E′ and the E′/E ratio of diastolic function.

Regarding infarct size measurement, the hearts of the subjects of the first group 106, the second group 108, the third group 110, and the fourth group 112 were fixed with 4% formalin for 24 hours post-experiment. The transverse tissue sections of the heart were stained with 1.5% 2,3,5-triphenyl tetrazolium chloride (TTC) for a time period of about 30 minutes at a temperature of about 37° C. and was quantified for the extent of myocardial tissue damage using ImageJ analysis software (NIH, USA).

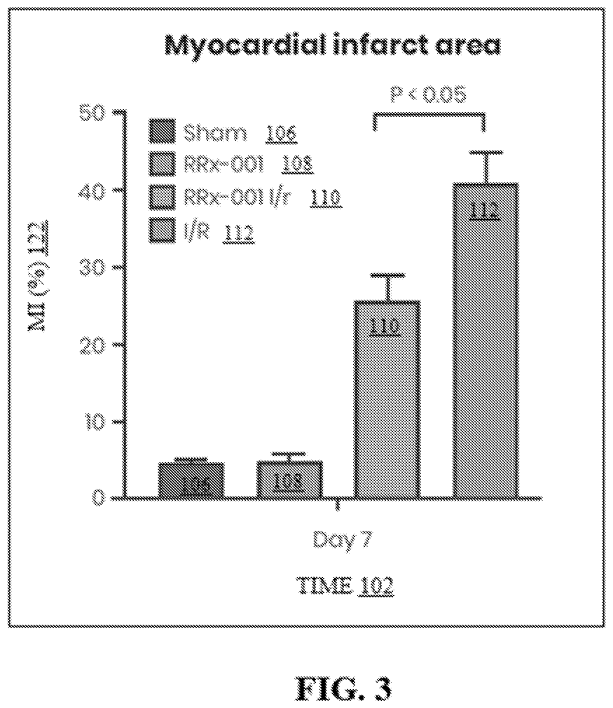

FIG. 3 depicts a graph showing myocardial infarct area (or MI) or area at risk for various groups of Example 1, according to at least some embodiments disclosed herein. In FIG. 3, the x-axis 102 is associated with time (at Day 7) and a y-axis 122 is associated with MI (which is measured as a percentage). As shown in FIG. 3, RRx-001 (e.g., the second group 108) ameliorated I/R-induced cardiac tissue damage. FIG. 1-FIG. 3 provide a strong rationale that RRx-001 treatment ameliorates the progression of adverse cardiac remodeling following MI.

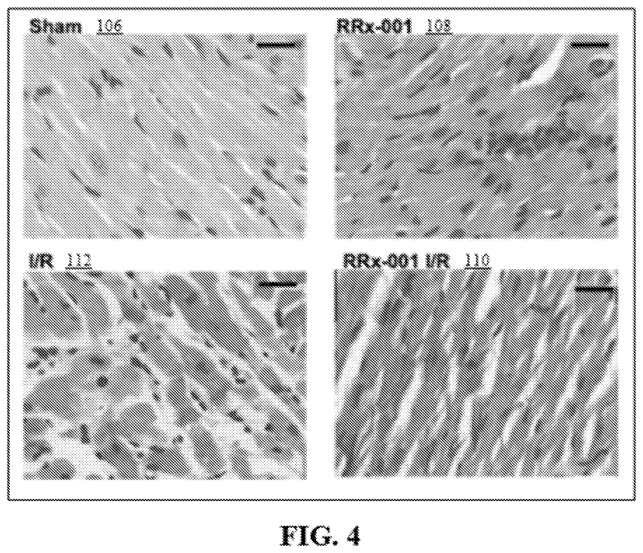

FIG. 4 depicts images showing a degree of damaged myocardium and fibrotic scar tissue of Example 1, according to at least some embodiments disclosed herein. As shown in FIG. 4, RRx-001 preserved myocardial tissue and reduced cardiomyocytes necrosis, inflammatory cell infiltration, and fibrous hyperplasia in the subjects of the second group 108.

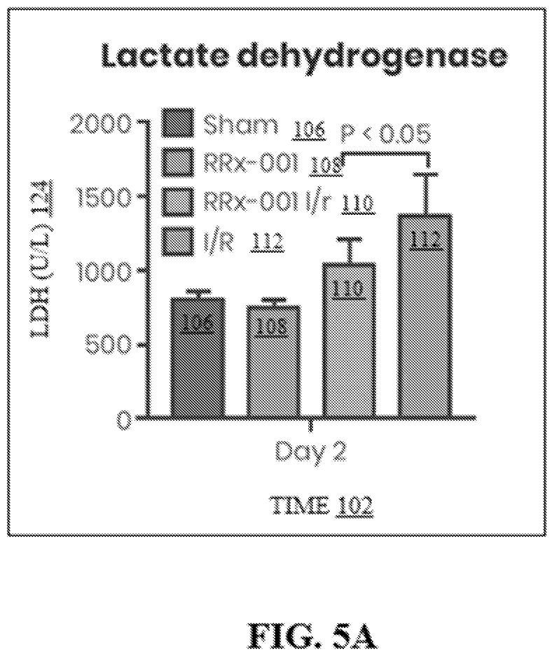

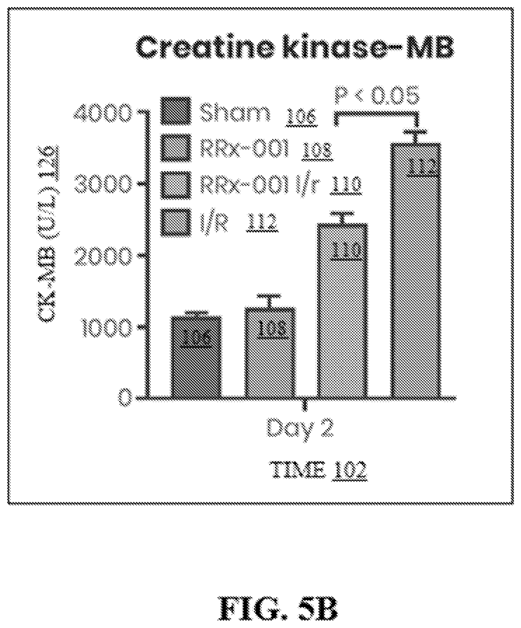

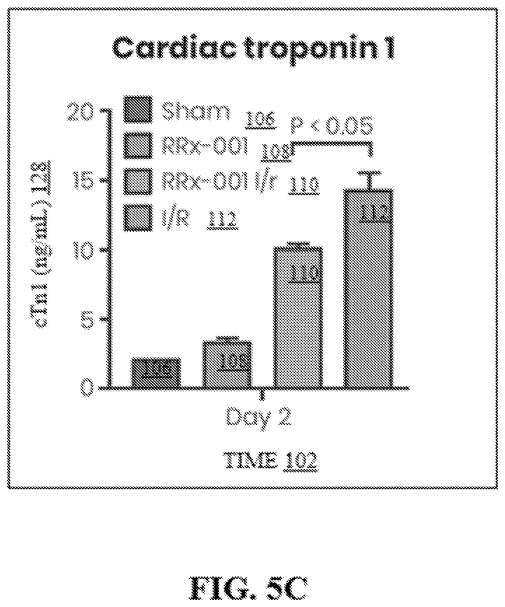

FIG. 5A, FIG. 5B, and FIG. 5C depict graphs showing RRx-001 reduced levels of serum biomarkers post-I/R. Specifically, FIG. 5A depicts a graph showing RRx-001 reduced levels of lactate dehydrogenase (LDH) post-IR of Example 1, FIG. 5B depicts a graph showing RRx-001 reduced levels of creatine phosphokinase-MB (CK-MB) post-IR of Example 1, and FIG. 5C depicts a graph showing RRx-001 reduced levels of cardiac Troponin 1 (cTn1) post-IR of Example 1, according to at least some embodiments disclosed herein.

Each of FIG. 5A, FIG. 5B, and FIG. 5C depict the x-axis 102 associated with time (measured at Day 2). FIG. 5A depicts a y-axis 124 associated with a level of LDH (measured as U/L). FIG. 5B depicts a y-axis 126 associated with a level of CK-MB (measured as U/L). FIG. 5C depicts a y-axis 128 associated with a level of cTn1 (measured as ng/mL). As shown in FIG. 5A, FIG. 5B, and FIG. 5C, the serum levels of LDH, CK-MB, and cTn1 decreased significantly for RRx-001 treated subjects (e.g., the subjects of the second group 108).

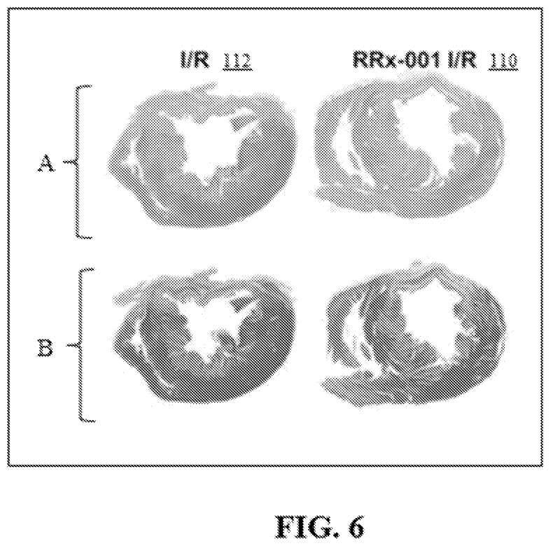

Histopathological examination occurred, where the hearts of the subjects were fixed in formalin and were made into tissue blocks after embedding in paraffin. The blocks were then cut into 5-μm sections and stained with hematoxylin and eosin (H&E). The sections were then examined under a light microscope (Olympus, Japan) for the pathological changes at 40× magnification. FIG. 6 depicts images showing H&E staining and Masson's trichrome staining of collagen fibers of Example 1, according to at least some embodiments disclosed herein. Row A in FIG. 6 displays H&E staining and Row B in FIG. 6 depicts Masson's trichrome staining of collagen fibers (scale bar=50 m). As shown in FIG. 6, RRx-001 reduced atrial fibrosis and collagen in the rat subjects that were subjected to I/R. Histology confirmed the reduction on the degree of LV fibrosis in the RRx-001 treatment.

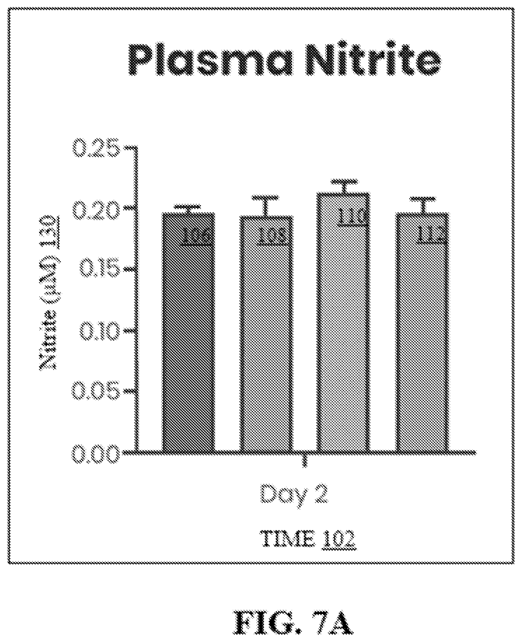

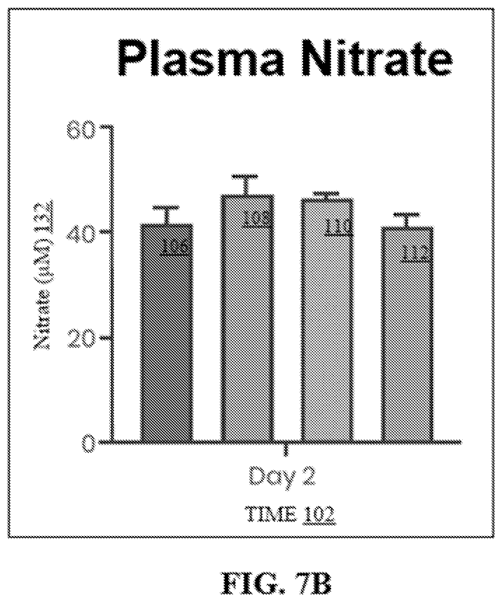

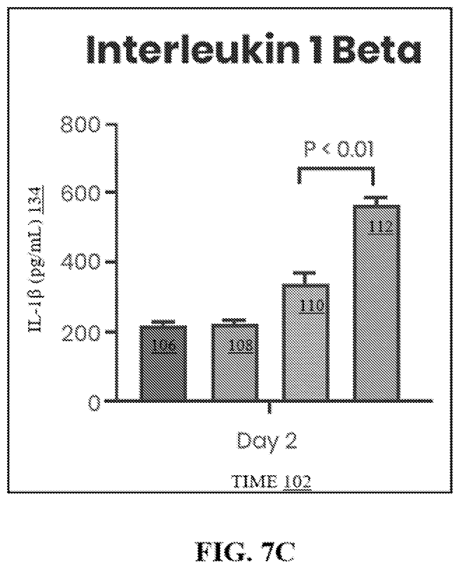

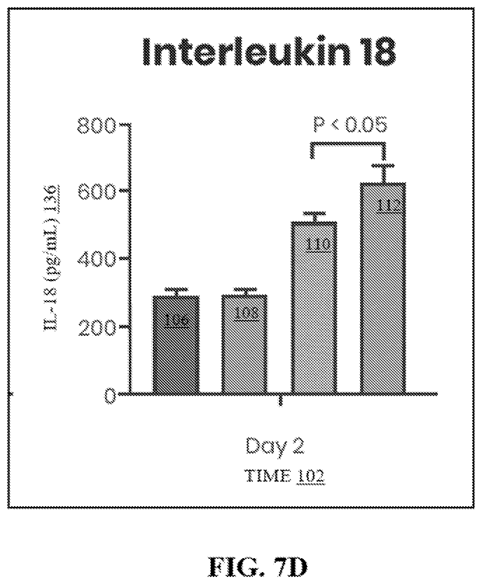

Changes in NOx species (e.g., plasma nitrite and nitrate), interleukin 1 beta (IL-1β), and interleukin 18 (IL-18) in species peripheral blood are depicted in FIG. 7A-FIG. 7D. Specifically, FIG. 7A depicts a graph showing changes in plasma nitrite in peripheral blood of the groups of Example 1, FIG. 7B depicts a graph showing changes in plasma nitrate in peripheral blood of the groups of Example 1, FIG. 7C depicts a graph showing changes in an IL-1β level in peripheral blood of the groups of Example 1, and FIG. 7D depicts a graph showing changes in an IL-18 level in peripheral blood of the groups of Example 1, according to at least some embodiments disclosed herein. Each of FIG. 7A-FIG. 7D depicts the x-axis 102 associated with time (measured at Day 2). A y-axis 130 of FIG. 7A is associated with a nitrite level (measured in μM). A y-axis 132 of FIG. 7B is associated with a nitrate level (measured in μM). A y-axis 134 of FIG. 7C is associated with an IL-1β level (measured in pg/mL). A y-axis 136 of FIG. 7D is associated with an IL-18 level (measured in pg/mL).

As shown in FIG. 7A and FIG. 7B, there were no significant changes in the NO species (nitrite and nitrate) detected at Day 2. As shown in FIG. 7C and FIG. 7D, the IL-1β and the IL-18 levels measured by an enzyme-linked immunosorbent assay (ELISA) were increased after I/R (e.g., the fourth group 112) and reduced in the subjects treated with RRx-001 (e.g., the second group 108).

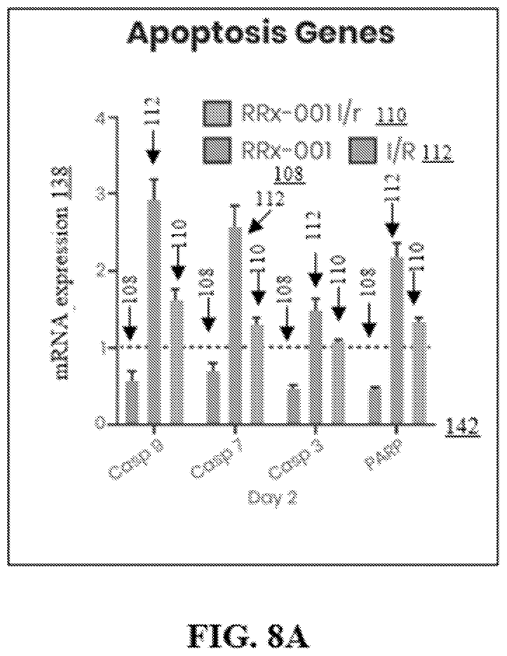

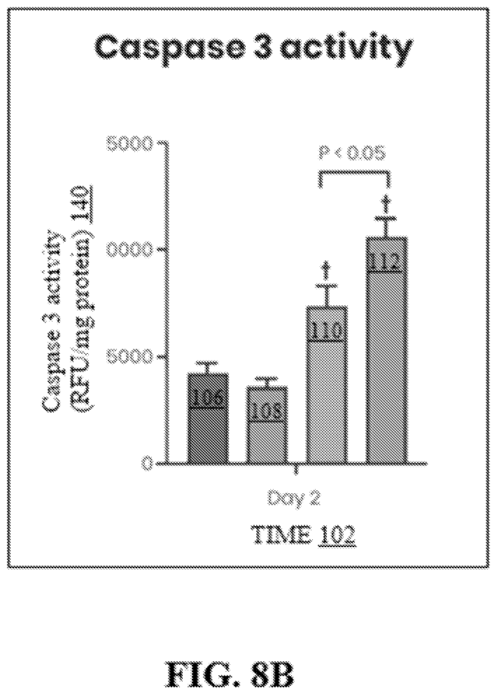

FIG. 8A depicts a graph showing mRNA expression of apoptosis genes in Example 1 and FIG. 8B depicts a graph showing caspase 3 activity for groups associated with Example 1, according to at least some embodiments disclosed herein. In FIG. 8A, an x-axis 142 is associated with various apoptosis genes (e.g., CASP9, CASP 7, CASP 3, and PARP) at Day 2 and a y-axis 138 is associated with mRNA expression (e.g., a fold change relative to sham). FIG. 8B includes the x-axis 102 associated with time (at Day 2) and a y-axis 140 associated with caspase 3 activity (measured as RFU/mg protein). Apoptosis and inflammation are the key pathological features of cardiac I/R injury. As shown in FIG. 8A and FIG. 8B, RRx-001 (e.g., the second group 108) reduced the I/R-associated changes in apoptotic caspases mRNA expression and reduced caspase 3 enzyme activity.

As shown in the Figures associated with Example 1, treatment with RRx-001 after reperfused myocardial infarction (or MI) decreased infarct expansion and scarring, increased LV systolic function to levels approaching normal values, and reduced myocardial hypertrophy. No hypotensive effects were observed. Compared with the controls, infarcted subject rats treated with RRx-001 significantly attenuated myocardial messenger RNA and protein levels of NF-κB, NLRP3 inflammasome, mature caspase-1, and IL-1β. As such, the methods described herein demonstrate that RRx-001 reduces infarct size, promoting administration of RRx-001 against ischemia/reperfusion injury.

These beneficial effects of RRx-001 are attributed to reducing inflammation and suppressing apoptosis. The MI causes injury by reducing the blood supply to the tissues and triggering extensive inflammatory during reperfusion. The RRx-001 treatment against ischemic heart disease alleviates adverse cardiac remodeling and improves contractile dysfunction. These results provide support that RRx-001 treatment may facilitate the progression of adverse cardiac remodeling following MI.

Although not wishing to be bound by a particular theory, the mechanism underlying RRx-001 treatment significantly improving cardiac function and LV fibrosis in subject rats with MI involves inhibition of NLRP3 inflammasome activation, NO supplementation, and suppression of the apoptosis signaling pathway. These results constitute a novel upstream therapy for MI.

### Other Embodiments

This application refers to various published patent applications, journal articles, and other publications, each of which is incorporated herein by reference.

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

What is claimed is:

1. A method for treating or preventing a condition related to myocardial infarction and infarct expansion, extension and/or dilatation and adverse remodeling following a cardiovascular disorder incident in a subject, the method comprising administering an effective amount of RRx-001, or a pharmaceutically acceptable salt thereof, to the subject at a time period post-infarction.

2. The method of claim 1, wherein the effective amount of RRx-001, or a pharmaceutically acceptable salt thereof, is a therapeutically effective amount of the RRx-001, or a pharmaceutically acceptable salt thereof.

3. The method of claim 2, wherein the therapeutically effective amount of the RRx-001, or a pharmaceutically acceptable salt thereof, is in a range of about 0.1 g/kg to about 10 mg/kg.

4. The method of claim 2, wherein the therapeutically effective amount of the RRx-001, or a pharmaceutically acceptable salt thereof, is in a range of about 1 g to about 5 mg/kg.

5. The method of claim 2, wherein the therapeutically effective amount of the RRx-001, or a pharmaceutically acceptable salt thereof, is in a range of about 0.05 mg/kg to about 10 mg/kg.

6. The method of claim 5, wherein the therapeutically effective amount of the RRx-001, or a pharmaceutically acceptable salt thereof, is in a range of about 0.1 mg/kg to about 10 mg/kg.

7. The method of claim 6, wherein the therapeutically effective amount of the RRx-001, or a pharmaceutically acceptable salt thereof, is in a range of about 0.1 mg/kg to about 5 mg/kg.

8. The method of claim 6, wherein the therapeutically effective amount of the RRx-001, or a pharmaceutically acceptable salt thereof, is in a range of about 0.5 mg/kg to about 3 mg/kg.

9. The method of claim 6, wherein the therapeutically effective amount of the RRx-001, or a pharmaceutically acceptable salt thereof, is in a range of about 0.1 mg/kg to about 1.5 mg/kg.

10. The method of claim 6, wherein the therapeutically effective amount of the RRx-001, or a pharmaceutically acceptable salt thereof, is in a range of about 0.1 mg/kg to about 0.35 mg/kg.

11. The method of claim 6, wherein the therapeutically effective amount of the RRx-001, or a pharmaceutically acceptable salt thereof, is in a range of about 0.35 mg/kg to about 0.75 mg/kg.

12. The method of claim 6, wherein the therapeutically effective amount of the RRx-001, or a pharmaceutically acceptable salt thereof, is in a range of about 0.75 mg/kg to about 1 mg/kg.

13. The method of any one of claims 1-12, wherein the time period post-infarction is in a range of about 1 hour to about three months.

14. The method of claim 13, wherein the time period post-infarction is in the range of about 1 hour to about one month.

15. The method of any one of claims 1-14, wherein the subject is a mammal subject.

16. The method of claim 15, wherein the mammal subject is a human subject.

17. The method of claim 15, wherein the mammal subject is an animal subject.

18. The method of any one of claims 1-17, wherein administering the effective amount of RRx-001, or a pharmaceutically acceptable salt thereof, occurs via oral administration, transdermal administration, administration by inhalation, nasal administration, topical administration, intra-aural administration, rectal administration, intravenous administration, elution from a stent, intramuscular administration, subcutaneous administration, or intraperitoneal administration, or combinations thereof.

19. The method of any one of claims 1-17, wherein administering the effective amount of the RRx-001, or a pharmaceutically acceptable salt thereof, occurs parenterally.

20. The method of any one of claims 1-17, wherein administering the effective amount of the RRx-001, or a pharmaceutically acceptable salt thereof, occurs orally.

21. The method of any one of claims 1-17, wherein administering the effective amount of the RRx-001, or a pharmaceutically acceptable salt thereof, occurs percutaneously.

22. The method of any one of claims 1-17, wherein administering the effective amount of the RRx-001, or a pharmaceutically acceptable salt thereof, occurs epicardially.

23. The method of any one of claims 1-17, wherein administering the effective amount of the RRx-001, or a pharmaceutically acceptable salt thereof, occurs endocardially.

24. The method of any one of claims 1-17, wherein administering the effective amount of RRx-001, or a pharmaceutically acceptable salt thereof, occurs via intravenous administration.

25. The method of any one of claims 1-17, wherein administering the effective amount of RRx-001, or a pharmaceutically acceptable salt thereof, occurs via direct intratumoral injection.

26. The method of any of claims 2-25, wherein administering the therapeutically effective amount of RRx-001, or a pharmaceutically acceptable salt thereof, is performed via a single administration.

27. The method of any one of claims 2-25, wherein administering the therapeutically effective amount of RRx-001, or a pharmaceutically acceptable salt thereof, is performed via at least two administrations.

28. The method of claim 27, wherein administering the therapeutically effective amount of RRx-001, or a pharmaceutically acceptable salt thereof, is performed at a frequency of between about 1 time per hour and about 1 time per month during the time period.

29. The method of claim 27, wherein administering the therapeutically effective amount of RRx-001, or a pharmaceutically acceptable salt thereof, is performed at a frequency of between about 4 times per day and about 1 time per week during the time period.

30. The method of claim 27, wherein administering the therapeutically effective amount of RRx-001, or a pharmaceutically acceptable salt thereof, is performed at a frequency of between about 2 times per day and about 3 times per week during the time period.

31. The method of claim 27, wherein administering the therapeutically effective amount of RRx-001, or a pharmaceutically acceptable salt thereof, is performed at a frequency of about one time a day during the time period.

32. The method of claim 27, wherein administering the therapeutically effective amount of RRx-001, or a pharmaceutically acceptable salt thereof, is performed at a frequency of between about 1 time per day and about 2 times per week during the time period.

33. The method of any one of claims 1-32, wherein the cardiovascular disorder incident is selected from the group consisting of: pulmonary hypertension, systemic hypertension, angina, Cardiac syndrome X, acute myocardial infarction, stroke, aneurysm, atrial fibrillation, congestive heart failure, dilated myocardiopathy, a heart valve disease, left ventricular hypertrophy, atrial myxoma, a congenital heart disease, hypercholesterolemia, diabetes mellitus, peripheral artery disease, Raynaud's disease, pulmonary embolism, and intravascular thrombosis.

34. The method of claim 33, wherein the cardiovascular disorder incident comprises angina, and wherein the angina comprises Prinzmetal's angina.

35. The method of claim 33, wherein the cardiovascular disorder incident comprises the heart valve disease, and wherein the heart valve disease is selected from the group consisting of: mitral rheumatic stenosis, infectious endocarditis, and marantic endocarditis.

36. The method of claim 33, wherein the cardiovascular disorder incident comprises the congenital heart disease, and wherein the congenital heart disease is selected from the group consisting of: patent foramen ovale, atrial septal aneurysm, and a ventricular septal defect.

37. The method of claim 33, wherein the cardiovascular disorder incident is acute myocardial infarction.

38. The method of any one of claims 1-37, wherein the condition related to myocardial infarction and infarct expansion and/or extension is selected from the group consisting of:
atherosclerosis, coronary artery spasm, a chronic disease, thrombosis, an embolism, and stenosis.

39. The method of claim 38, wherein the condition related to myocardial infarction and infarct expansion and/or extension comprises the chronic disease, and wherein the chronic disease is selected from the group consisting of: human immunodeficiency virus (HIV), diabetes, hypothyroidism, hyperthyroidism, hemochromatosis, and amyloidosis.

40. The method of claim 1, wherein the method prevents cardiac dilatation after myocardial infarction.

41. The method of any one of claims 1-40, wherein the method improves cardiac function after myocardial infarction.

42. The method of any one of claims 1-40, wherein the method prevents heart failure after myocardial infarction.

43. The method of any one of claims 2-42, wherein administering the therapeutically effective amount of the RRx-001, or a pharmaceutically acceptable salt thereof, improves cardiac function in the subject experiencing at least one of systolic and/or diastolic dysfunction of the left ventricle (LV) and/or right ventricle (RV), myocardial hypertrophy, myocardial dilatation, myocardial fibrosis, depressed myocardial shortening, diminished cardiac contractility, reduced stroke volume, diminished fractional shortening, diminished ejection fraction, increased LV end diastolic pressure, increased ventricular wall stress, increased ventricular wall tension, increased LV systolic volume, increased LV diastolic volume, and increased thickness of the posterior wall of the heart.

44. The method of any one of claims 1-40, wherein the method prevents cardiac remodeling after myocardial infarction.

45. The method of claim 44, wherein the cardiac remodeling results from an acute coronary syndromes, cardiac fibrosis, a pulmonary embolism, pericarditis, myocarditis, coronary vasospasm, hypertension, myocardial infarction, a valvular heart disease, left ventricular dysfunction, cardiac ischemia, angina, tachycardia, bradycardia, hypertrophic cardiomyopathy, dilated myocardiopathy, heart valve disease, left ventricular hypertrophy, atrial myxoma, or a congenital heart disease.

46. The method of claim 45, wherein the cardiac remodeling results from the acute coronary syndrome, and wherein the acute coronary syndrome is selected from the group consisting of: an ST elevation myocardial infarction and a non-ST elevation acute coronary syndrome (NSTE-ACS).

47. The method of claim 45, wherein the cardiac remodeling results from the valvular heart disease, and wherein the valvular heart disease results from a mechanical prosthetic valve, mitral rheumatic stenosis, infectious endocarditis, or marantic endocarditis.

48. The method of claim 45, wherein the cardiac remodeling results from a congenital heart disease, and wherein the congenital heart disease is selected from the group consisting of: patent foramen ovale, an atrial septal aneurysm, and a ventricular septal defect.

49. The method of any one of claims 2-48, wherein administering the therapeutically effective amount of RRx-001, or a pharmaceutically acceptable salt thereof, preserves heart ejection fraction and fractional shortening and mitigates an increase in left ventricular end-systolic diameter (LVESD) and left ventricular end-diastolic diameter (LVEDD).

50. The method of any one of claims 2-49, wherein administering the therapeutically effective amount of RRx-001, or a pharmaceutically acceptable salt thereof, preserves myocardial tissue.

51. The method of any one of claims 2-50, wherein administering the therapeutically effective amount of RRx-001, or a pharmaceutically acceptable salt thereof, reduces a level of at least one serum biomarker in the subject post-infarction.

52. The method of claim 51, wherein each serum biomarker of the at least one serum biomarker is selected from the group consisting of: lactate dehydrogenase (LDH), creatine phosphokinase-MB (CK-MB), and cardiac Troponin 1 (cTn1).

53. The method of any one of claims 2-52, wherein administering the therapeutically effective amount of RRx-001, or a pharmaceutically acceptable salt thereof, reduces at least one of: infarct expansion, infarct scarring, myocardial hypertrophy, atrial fibrosis, collagen, IL-1β level, IL-18 level, caspase 3 enzyme activity, cardiomyocytes necrosis, inflammatory cell infiltration, and fibrous hyperplasia in the subject.

54. The method of any of claims 2-53, wherein the therapeutically effective amount of the RRx-001, or a pharmaceutically acceptable salt thereof, is administered as a composition comprising a blood product.

55. The method of claim 54, wherein the blood product comprises erythrocyte cells.

56. The method of claim 55, wherein the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis.

57. The method of claim 54, wherein the blood product is a mixture of packed red blood cells.

58. The method of claim 54, wherein the blood product is whole blood.

59. The method of claim 58, wherein the whole blood is autologous whole blood or donor-matched allogenic whole blood.

60. The method of any one of claims 1-59, further comprising:
administering an agent prior to, concurrently with, or subsequent administering the effective amount of RRx-001, or a pharmaceutically acceptable salt thereof.

61. The method of claim 60, wherein the agent is selected from the group consisting of: a statin, an angiotensin-converting-enzyme (ACE) inhibitor, aspirin, oxygen, a nitric oxide donor, a drug-eluting stent, an anticoagulant or antiplatelet therapy, a phosphodiesterase-5 inhibitor, a calcium channel blocker, an angiotensin II receptor blocker (ARB), a beta blocker, an aldosterone antagonist, a loop diuretic, an epigenetic inhibitor, and a nitrite.

62. The method of any one of claims 60-61, wherein an amount of the agent is in a range of about 1 mg to about 50 mg.

63. The method of claim 62, wherein the amount of the agent is in the range of about 1 mg to about 15 mg.

64. The method of any one of claims 60-63, wherein administering the agent prior to, concurrently with, or subsequent administering the effective amount of RRx-001, or a pharmaceutically acceptable salt thereof, prevents at least one side effect associated with the agent.

65. The method of any one of claims 60-63, wherein administering the agent prior to, concurrently with, or subsequent administering the effective amount of RRx-001, or a pharmaceutically acceptable salt thereof, reduces at least one side effect associated with the agent.

66. A pharmaceutical composition for treating or preventing a condition related to myocardial infarct expansion, extension, and/or dilatation and adverse remodeling following a cardiovascular disorder incident in a subject, the pharmaceutical composition comprising an effective amount of RRx-001, or a pharmaceutically acceptable salt thereof.

67. The pharmaceutical composition of claim 66, wherein the effective amount of RRx-001, or a pharmaceutically acceptable salt thereof, is a therapeutically effective amount of the RRx-001, or a pharmaceutically acceptable salt thereof.

68. The pharmaceutical composition of claim 67, wherein the therapeutically effective amount of the RRx-001, or a pharmaceutically acceptable salt thereof, is in a range of about 0.1 g/kg to about 10 mg/kg.

69. The pharmaceutical composition of claim 67, wherein the therapeutically effective amount of the RRx-001, or a pharmaceutically acceptable salt thereof, is in a range of about 1 g to about 5 mg/kg.

70. The pharmaceutical composition of claim 67, wherein the therapeutically effective amount of the RRx-001, or a pharmaceutically acceptable salt thereof, is in a range of about 0.05 mg/kg to about 10 mg/kg.

71. The pharmaceutical composition of claim 70, wherein the therapeutically effective amount of the RRx-001, or a pharmaceutically acceptable salt thereof, is in a range of about 0.1 mg/kg to about 10 mg/kg.

72. The pharmaceutical composition of claims 66-71, wherein the time period post-infarction is in a range of about 1 hour to about three months.

73. The pharmaceutical composition of claim 72, wherein the time period post-infarction is in the range of about 1 hour to about one month.

74. The pharmaceutical composition of any one of claims 66-73, wherein the subject is a mammal subject.

75. The pharmaceutical composition of claim 74, wherein the mammal subject is a human subject.

76. The pharmaceutical composition of claim 74, wherein the mammal subject is an animal subject.

77. The pharmaceutical composition of any one of claims 66-76, wherein the cardiovascular disorder incident is selected from the group consisting of: pulmonary hypertension, systemic hypertension, angina, Cardiac syndrome X, acute myocardial infarction, stroke, aneurysm, atrial fibrillation, congestive heart failure, dilated myocardiopathy, a heart valve disease, left ventricular hypertrophy, atrial myxoma, a congenital heart disease, peripheral artery disease, Raynaud's disease, pulmonary embolism, and intravascular thrombosis.

78. The pharmaceutical composition of claim 77, wherein the cardiovascular disorder incident comprises angina, and wherein the angina comprises Prinzmetal's angina.

79. The pharmaceutical composition of claim 77, wherein the cardiovascular disorder incident comprises the heart valve disease, and wherein the heart valve disease is selected from the group consisting of: mitral rheumatic stenosis, infectious endocarditis, and marantic endocarditis.

80. The pharmaceutical composition of claim 77, wherein the cardiovascular disorder incident comprises the congenital heart disease, and wherein the congenital heart disease is selected from the group consisting of: patent foramen ovale, atrial septal aneurysm, and a ventricular septal defect.

81. The pharmaceutical composition of claim 77, wherein the cardiovascular disorder incident is acute myocardial infarction.

82. The pharmaceutical composition of any one of claims 66-81, wherein the condition related to myocardial infarction and infarct expansion and/or extension is selected from the group consisting of: atherosclerosis, coronary artery spasm, a chronic disease, thrombosis, an embolism, and stenosis.

83. The pharmaceutical composition of claim 82, wherein the condition related to myocardial infarction and infarct expansion and/or extension comprises the chronic disease, and wherein the chronic disease is selected from the group consisting of: human immunodeficiency virus (HIV), diabetes, hypothyroidism, hyperthyroidism, hemochromatosis, and amyloidosis.

84. The pharmaceutical composition of any one of claims 67-83, wherein administration of the therapeutically effective amount of the RRx-001, or a pharmaceutically acceptable salt thereof, improves cardiac function in the subject experiencing at least one of systolic and/or diastolic dysfunction of the left ventricle (LV) and/or right ventricle (RV), myocardial hypertrophy, myocardial dilatation, myocardial fibrosis, depressed myocardial shortening, diminished cardiac contractility, reduced stroke volume, diminished fractional shortening, diminished ejection fraction, increased LV end diastolic pressure, increased ventricular wall stress, increased ventricular wall tension, increased LV systolic volume, increased LV diastolic volume, and increased thickness of the posterior wall of the heart.

85. The pharmaceutical composition of any one of claims 66-84, wherein the pharmaceutical composition prevents cardiac remodeling after myocardial infarction.

86. The pharmaceutical composition of claim 85, wherein the cardiac remodeling results from an acute coronary syndromes, cardiac fibrosis, a pulmonary embolism, pericarditis, myocarditis, coronary vasospasm, hypertension, myocardial infarction, a valvular heart disease, left ventricular dysfunction, cardiac ischemia, angina, tachycardia, bradycardia, hypertrophic cardiomyopathy, dilated myocardiopathy, heart valve disease, left ventricular hypertrophy, atrial myxoma, or a congenital heart disease.

87. The pharmaceutical composition of claim 86, wherein the cardiac remodeling results from the acute coronary syndrome, and wherein the acute coronary syndrome is selected from the group consisting of: an ST elevation myocardial infarction and a non-ST elevation acute coronary syndrome (NSTE-ACS).

88. The pharmaceutical composition of claim 86, wherein the cardiac remodeling results from the valvular heart disease, and wherein the valvular heart disease results from a mechanical prosthetic valve, mitral rheumatic stenosis, infectious endocarditis, or marantic endocarditis.

89. The pharmaceutical composition of claim 86, wherein the cardiac remodeling results from a congenital heart disease, and wherein the congenital heart disease is selected from the group consisting of: patent foramen ovale, an atrial septal aneurysm, and a ventricular septal defect.

90. The pharmaceutical composition of any one of claims 66-89, wherein administration of the therapeutically effective amount of RRx-001, or a pharmaceutically acceptable salt thereof, preserves heart ejection fraction and fractional shortening, preserves myocardial tissue, and mitigates an increase in left ventricular end-systolic diameter (LVESD) and left ventricular end-diastolic diameter (LVEDD).

91. The pharmaceutical composition of any one of claims 67-90, wherein administration of the therapeutically effective amount of RRx-001, or a pharmaceutically acceptable salt thereof, reduces a level of at least one serum biomarker in the subject post-infarction.

92. The pharmaceutical composition of claim 91, wherein each serum biomarker of the at least one serum biomarker is selected from the group consisting of: lactate dehydrogenase (LDH), creatine phosphokinase-MB (CK-MB), and cardiac Troponin 1 (cTn1).

93. The pharmaceutical composition of any one of claims 67-92, wherein administration of the therapeutically effective amount of RRx-001, or a pharmaceutically acceptable salt thereof, reduces at least one of: infarct expansion, infarct scarring, myocardial hypertrophy, atrial fibrosis, collagen, IL-1β level, IL-18 level, caspase 3 enzyme activity, cardiomyocytes necrosis, inflammatory cell infiltration, and fibrous hyperplasia in the subject.

94. The pharmaceutical composition of any one of claims 67-93, wherein the pharmaceutical composition further comprises a blood product.

95. The pharmaceutical composition of claim 94, wherein the blood product comprises erythrocyte cells.

96. The pharmaceutical composition of claim 95, wherein the erythrocyte cells have not undergone any manipulation selected from the group consisting of genetic modification, electroporation, conjugation through biotin, conjugation to a cell-penetrating peptide, conjugation to hemoglobin, dimethyl sulfoxide osmotic pulse, endocytosis and hypotonic preswelling, hypotonic dilution, and hypo-osmotic dialysis.

97. The pharmaceutical composition of claim 94, wherein the blood product is a mixture of packed red blood cells.

98. The pharmaceutical composition of claim 94, wherein the blood product is whole blood.

99. The pharmaceutical composition of claim 98, wherein the whole blood is autologous whole blood or donor-matched allogenic whole blood.

100. The pharmaceutical composition of any one of claims 66-99, wherein the pharmaceutical composition further comprises an agent.

101. The pharmaceutical composition of claim 100, wherein the agent is selected from the group consisting of: a statin, an angiotensin-converting-enzyme (ACE) inhibitor, aspirin, oxygen, a nitric oxide donor, a drug-eluting stent, an anticoagulant or antiplatelet therapy, a phosphodiesterase-5 inhibitor, a calcium channel blocker, an angiotensin II receptor blocker (ARB), a beta blocker, an aldosterone antagonist, a loop diuretic, an epigenetic inhibitor, and a nitrite.

102. The pharmaceutical composition of any one of claims 100-101, wherein an amount of the agent is in a range of about 1 mg to about 50 mg.

103. The pharmaceutical composition of claim 102, wherein the amount of the agent is in the range of about 1 mg to about 15 mg.
